(19) United States
(12) Patent Application Publication
(10) Pub. No.: US 2020/0121645 A1
(43) Pub. Date: Apr. 23, 2020

(54) COMPOSITION FOR PREVENTING OR TREATING EGFR-MUTANT NON-SMALL CELL LUNG CANCER

(71) Applicants: THE ASAN FOUNDATION, Seoul (KR); University of Ulsan Foundation For Industry Cooperation, Ulsan (KR)

(72) Inventors: Jaekyoung SON, Seoul (KR); Jin Kyung RHO, Seoul (KR); Jae Cheol LEE, Seoul (KR)

(73) Assignees: THE ASAN FOUNDATION, Seoul (KR); University of Ulsan Foundation For Industry Cooperation, Ulsan (KR)

(21) Appl. No.: 16/274,749

(22) Filed: Feb. 13, 2019

(30) Foreign Application Priority Data
Oct. 23, 2018 (KR) 10-2018-0126479

(51) Int. Cl. A61K 31/4025; A61P 35/00; A61K 9/00; A23L 29/00

(52) U.S. Cl. CPC A61K 31/4025; A61P 35/00; A23V 2200/308; A61K 9/0065; A23L 29/045; A61K 9/006

(57) ABSTRACT

Disclosed is a composition and method for preventing, ameliorating or treating an EGFR-mutant non-small cell lung cancer including a c-Jun N-terminal kinase (JNK) activator as an active ingredient. The composition significantly reduces the level of EGFR in EGFR-mutant non-small cell lung cancer cells, inducing apoptosis. Therefore, the composition is suitable for preventing, ameliorating or treating non-small cell lung cancers in subjects in need thereof. Particularly, the composition is effective in treating and preventing non-small cell lung cancers, which are difficult to effectively treat and prevent with gefitinib or erlotinib. Also disclosed is a composition and method for inhibiting the resistance of a non-small cell lung cancer to an EGFR tyrosine kinase inhibitor including a c-Jun N-terminal kinase (JNK) activator as an active ingredient. The inhibitory composition effectively overcomes resistance to EGFR tyrosine kinase inhibitors.

### CROSS-REFERENCE TO RELATED APPLICATIONS

This application claims priority under 35 U.S.C. § 119 to Korean Patent Application No. 10-2018-0126479 filed on Oct. 23, 2018 in the Korean Intellectual Property Office, the disclosure of which is incorporated herein by reference in its entirety.

### BACKGROUND OF THE INVENTION

### 1. Field of the Invention

The present invention relates to a composition and method for preventing, ameliorating or treating an EGFR-mutant non-small cell lung cancer including a c-Jun N-terminal kinase (JNK) activator as an active ingredient.

### 2. Description of the Related Art

The epidermal growth factor receptor (EGFR), a member of the HER family of receptor tyrosine kinases, mediates cell proliferation, angiogenesis, invasion, and metastasis (Harari P M, et al. J Clin Oncol. 2007; 25:4057-65; Yarden Y, et al. Nat Rev Mol Cell Biol. 2001; 2:127-37). Aberrant expression of EGFR is frequently observed in multiple tumor types and is known to have a strong oncogenic potential (Hirsch F R, et al. J Clin Oncol. 2003; 21:3798-807; Rubin Grandis J, et al. J Natl Cancer Inst. 1998; 90:824-32).

First-generation EGFR-tyrosine kinase inhibitors (TKI) such as gefitinib and erlotinib reversibly bind to the ATP cleft within the EGFR kinase domain to block autophosphorylation of EGFR (Mendelsohn J, et al. J Clin Oncol. 2003; 21:2787-99). Although these EGFR-TKIs were shown to be effective in patients with advanced non-small cell lung cancer (NSCLC) harboring EGFR activating mutations such as small in-frame deletions in exon 19 or the L858R missense mutation in exon 21, patients almost always develop resistance to these agents, most commonly through the acquisition of a secondary T790M mutation in EGFR exon 20 (Pao W, et al. PLoS Med. 2005; 2:e73). To date, there is no standard therapeutic option for patients with acquired resistance to reversible EGFR TKIs due to acquisition of EGFR T790M (Riely G J. J Thorac Oncol. 2008; 3:S146-9).

Afatinib (BIBW2992) is one of the second-generation irreversible EGFR-TKIs. In recent preclinical studies, afatinib was shown to have antitumor activity in NSCLCs with the EGFR T790M in vitro and in vivo. On the basis of these results, afatinib is expected to be a standard therapeutic option for patients with NSCLCs with EGFR T790M (Li D, et al. Oncogene. 2008; 27:4702-11). However, afatinib was more than 100-fold less potent in NSCLC cells harboring EGFR T790M mutation than in NSCLC cells with activating EGFR mutation (Takezawa K, et al. Mol Cancer Ther. 2010; 9:1647-56). It also showed limited efficacy in a recent phase III clinical study suggesting the necessity of developing a new strategy to improve the efficacy of afatinib (Miller V A, et al. Lancet Oncol. 2012; 13:528-38).

Therefore, there is a need to develop a new family of therapeutic agents that can effectively treat EGFR-mutant non-small cell lung cancers, particularly non-small cell lung cancers with acquired resistance to EGFR tyrosine kinase inhibitors.

### PRIOR ART DOCUMENTS

### Patent Documents

(Patent Document 001) KR 10-2017-0015848 A

### SUMMARY OF THE INVENTION

One object of the present invention is to provide a pharmaceutical composition for preventing or treating an EGFR-mutant non-small cell lung cancer including a c-Jun N-terminal kinase (JNK) activator as an active ingredient.

A further object of the present invention is to provide a composition for inhibiting the resistance of a non-small cell lung cancer to an EGFR tyrosine kinase inhibitor including a c-Jun N-terminal kinase (JNK) activator as an active ingredient.

Another object of the present invention is to provide a health functional food composition for preventing or ameliorating an EGFR-mutant non-small cell lung cancer including a c-Jun N-terminal kinase (JNK) activator as an active ingredient.

One aspect of the present invention provides a pharmaceutical composition for preventing or treating an EGFR-mutant non-small cell lung cancer including a c-Jun N-terminal kinase (JNK) activator as an active ingredient.

In one embodiment of the present invention, the JNK activator may be anisomycin or a derivative thereof.

In one embodiment of the present invention, the non-small cell lung cancer may harbor a deletion mutation in exon 19 of EGFR or a point mutation in exon 21 of EGFR.

In one embodiment of the present invention, the non-small cell lung cancer may further harbor a T790M mutation in exon 20 of EGFR.

In one embodiment of the present invention, the non-small cell lung cancer may be resistant to gefitinib or erlotinib.

In one embodiment of the present invention, the non-small cell lung cancer may be resistant to a reversible EGFR tyrosine kinase inhibitor due to a T790 M mutation in exon 20 of EGFR.

In one embodiment of the present invention, the pharmaceutical composition may further include a pharmaceutically acceptable carrier, excipient or diluent.

In one embodiment of the present invention, the pharmaceutical composition may be formulated into a liquid, powder, aerosol, injectable preparation, Ringer's solution, patch, capsule, pill, tablet, depot or suppository.

The non-small cell lung cancer may be selected from the group consisting of squamous cell carcinoma, adenocarcinoma, large cell carcinoma, adenosquamous carcinoma, and sarcomatoid carcinoma.

A further aspect of the present invention provides a composition for inhibiting the resistance of a non-small cell lung cancer to an EGFR tyrosine kinase inhibitor including a c-Jun N-terminal kinase (JNK) activator as an active ingredient.

In one embodiment of the present invention, the EGFR tyrosine kinase inhibitor may be gefitinib or erlotinib.

Another aspect of the present invention provides a health functional food composition for preventing or ameliorating an EGFR-mutant non-small cell lung cancer including a c-Jun N-terminal kinase (JNK) activator as an active ingredient.

The pharmaceutical composition of the present invention significantly reduces the level of EGFR in EGFR-mutant non-small cell lung cancer cells, inducing apoptosis. Therefore, the pharmaceutical composition of the present invention is suitable for preventing, ameliorating or treating non-small cell lung cancers. Particularly, the pharmaceutical composition of the present invention is effective in treating and preventing non-small cell lung cancers, which are difficult to effectively treat and prevent with gefitinib or erlotinib.

In addition, the inhibitory composition of the present invention effectively overcomes resistance to EGFR tyrosine kinase inhibitors.

Another aspect of the present application provides a method for the treatment, amelioration, or prevention of an EGFR-mutant non-small cell lung cancer in a subject in need thereof. In one embodiment, the method comprises administering to the subject a composition comprising a c-Jun N-terminal kinase (JNK) activator as an active ingredient. In one embodiment, the composition is a health functional food composition. In another embodiment, the composition is a pharmaceutical composition.

Another aspect of the present application provides a method for inhibiting or decreasing the resistance of a non-small cell lung cancer to an EGFR tyrosine kinase inhibitor in a subject in need thereof. In one embodiment, the method comprises administering to the subject a composition comprising a c-Jun N-terminal kinase (JNK) activator as an active ingredient. In one embodiment, the composition is a health functional food composition. In another embodiment, the composition is a pharmaceutical composition.

### BRIEF DESCRIPTION OF THE DRAWINGS

FIGS. 1a to 1n show that EGFR-mutant NSCLCs rely more heavily on aerobic glycolysis than do EGFR-WT NSCLCs. Specifically, FIG. 1a: Glycolysis metabolite pools in EGFR-mutant NSCLC were analyzed via LC/MS-MS. Western blot analysis confirmed EGFR knockdown; FIG. 1b: Summary of changes in glycolytic enzyme mRNA levels upon EGFR knockdown. The gene names for enzymes exhibiting significant changes are highlighted in gray bold; FIGS. 1c and 1d: EGFR-mutant NSCLCs expressing control (shGFP) or EGFR-targeting shRNA were plated in complete media and glucose uptake and lactate production were measured over time using a YSI 2300 STA Plus Glucose-Lactate Analyzer; FIG. 1e: Real-time glycolytic rates were determined using an extracellular flux analyzer. PC9 cells expressing control (shGFP) or EGFR-targeting shRNAs were sequentially treated with glucose (10 mM), oligomycin (1 μM), and 2DG (20 mM); FIGS. 1f and 1g: EGFR-WT and EGFR-mutant NSCLCs were plated in complete media and glucose uptake and lactate production were measured over time using a YSI 2300 STA plus Glucose-Lactate Analyzer; FIG. 1h: Real-time glycolytic rates were determined using an extracellular flux analyzer. Cells were sequentially treated with glucose (10 mM), oligomycin (1 μM), and 2DG (20 mM); FIGS. 1i, 1j, and 1k: SCID mice bearing established PC9, H1975, and A549 tumor cell xenografts were treated with 2DG (Materials and Methods). Tumor volumes were calculated on indicated days. Arrows, drug treatment cessation; FIG. 1l: EGFR-mutant NSCLCs were plated in complete media that was replaced the following day with glucose or glutamine-free medium, incubated for another 24 hours, and immunoblotted with indicated antibodies; FIG. 1m: Cells were treated with 2DG (10 mM) or BPTES (10 μM) for up to 48 hours and immunoblotted with indicated antibodies; and FIG. 1n: EGFR-mutant NSCLCs were plated in complete media that was replaced the following day with glucose-free medium or treated with 2DG (10 mM) for 24 hours and immunoblotted with indicated antibodies. Glc, glucose; Gln, glutamine; G6P, glucose-6-phosphate; FBP, fructose 1,6-bisphosphate; 3PG, 3-phosphoglycerate; PEP, phosphoenolpyruvate; PYR, pyruvate; LAC, lactate. *, P<0.05; **, P<0.01.

FIG. 2 shows that EGFR knockdown decreases glycolytic gene expression at the transcription level. The expression of glycolysis genes was determined by quantitative RT-PCR in PC9 cells expressing two independent shRNAs targeting the control shRNA(shGFP) or EGFR. p<0.05; **, p<0.01.

FIGS. 3a to 3l show that glucose deprivation renders EGFR-mutant NSCLCs more sensitive to cell growth and death. Specifically, FIGS. 3a to 3d: Cells were plated in complete media that was replaced the following day with glucose or glutamine-free medium, incubated for another 24 h, and assayed for cell growth; FIGS. 3e to 3h: EGFR-WT (A549 and H1299) and EGFR-mutant NSCLCs (PC9 and H1975) were plated in complete media. After 24 h, media was supplemented with glucose or glutamine-free medium. Cell death was assayed by annexin V/PI staining and flow cytometry; and FIGS. 3i to 3l: Cells were treated with 2DG (10 mM) or BPTES (10 μM) for 24 h and assayed for cell death by annexin V/PI staining and flowcytometry.

FIGS. 4a to 4i show that survival of EGFR-mutant NSCLCs requires glucose as a carbon source for the TCA cycle. Specifically, FIG. 4a: PC9 cells were plated in a complete media that was replaced the following day with glucose or glutamine-free medium, incubated for another 24 hours, and assayed for intracellular ATP; FIG. 4b: Oxygen consumption rates were measured with an extracellular flux analyzer. PC9 cells were plated in a complete media that was replaced the following day with glucose or glutamine-free medium. Cells were sequentially treated with oligomycin (2 μM), FCCP (5 μM), and rotenone (2 μM); FIG. 4c: TCA metabolite pools in PC9 cells expressing control (shGFP) or EGFR shRNAs (shEGFR) were analyzed via LC-MS/MS; FIGS. 4d and 4e: EGFR-mutant NSCLCs were plated in complete media that was replaced the following day with glucose-free medium supplemented with either methyl pyruvate (MPYR; 7 mM) or α-ketoglutarate (AKG; 7 mM), incubated for another 24 hours, and assayed for cell death by Annexin V/PI staining and flow cytometry; FIGS. 4f and 4g: EGFR-mutant NSCLCs were treated with 2DG (10 mM) in the presence of either MPYR (7 mM) or AKG (7 mM) and assayed for cell death by Annexin V/PI staining and flow cytometry; FIG. 4h: Cells were plated in complete media that was replaced the following day with MPYR-supplemented (7 mM) glucose-free medium, incubated for another 24 hours, and immunoblotted with indicated antibodies; and FIG. 4i: Cells were treated with 2DG (10 mM) and MPYR (7 mM) for 24 hours and immunoblotted with indicated antibodies. NS, not significant. *, P<0.05; **, P<0.01.

FIGS. 5a to 5c show that EGFR-mediated enhanced glycolysis is an important TCA cycle fuel source. Specifically, FIG. 5a: TCA metabolite pools in PC9 cells treated with 2DG (10 mM) or BPTES (10 μM) were analyzed via LC-MS/MS; FIG. 5b: Oxygen consumption rates in PC9 cells expressing control (shGFP) or EGFR shRNAs (shEGFRs) were measured with an extracellular flux analyzer. Cells were sequentially treated with oligomycin (2 μM), FCCP (5 μM), and rotenone (2 μM); and FIG. 5c: PC9 cells were plated in complete media that was replaced the following day with glucose-free medium supplemented with either methyl pyruvate (MPYR; 7 mM) or a-ketoglutarate (AKG; 7 mM), incubated for another 24 h, and assayed for intracellular ATP.

FIGS. 6a to 6d show that mitochondrial ATP production is necessary for EGFR stability. Specifically, FIGS. 6a and 6b: EGFR-mutant NSCLCs were treated with rotenone (Rot) for up to 48 h in the absence or presence of methyl-pyruvate (7 mM) and immunoblotted with indicated antibodies; and FIGS. c and d: EGFR-mutant NSCLCs were treated with Rot (5 μM) in the absence or presence of methyl-pyruvate and assayed for cell death by annexin V/PI staining and flow cytometry.

FIGS. 7a to 7l show that sustained JNK inactivity is required for EGFR stability. Specifically, FIG. 7a: EGFR-mutant NSCLCs were plated in complete media that was replaced the following day with glucose or glutamine-free medium, incubated for another 24 hours, and immunoblotted for JNK and P-JNK; FIG. 7b: EGFR-mutant NSCLCs were treated with rotenone and immunoblotted for JNK and P-JNK; FIG. 7c: EGFR-mutant NSCLCs were treated with anisomycin for 24 hours and immunoblotted with indicated antibodies; FIGS. 7d to 7g: EGFR-mutant and WT NSCLCs were treated with anisomycin for 24 hours and assayed for cell death by Annexin V/PI staining and flow cytometry; FIGS. 7h to 7i: SCID mice bearing established PC9 and H1975 tumor cell xenografts were treated with anisomycin (Materials and Methods). Tumor volumes were calculated on indicated days. Arrows, drug treatment cessation; FIGS. 7j to 7k: EGFR-mutant NSCLCs were cultured in complete or glucose-free medium with or without SP600125 (15 μM) and assayed for cell death by Annexin V/PI staining and flow cytometry; and FIG. 7l: EGFR-mutant NSCLCs were cultured in complete or glucose-free medium with or without SP600125 (15 μM) for 24 hours and immunoblotted for EGFR. ANS, anisomycin; SP, SP600125. **, P<0.01.

FIG. 8 shows that anisomycin treatment inhibits the EGFR signaling pathway and activates apoptosis via JNK activation in vivo. SCID mice bearing established PC9 and H1975 tumor cell xenografts were treated with anisomycin for 3 days and subjected to Western blotting for EGFR-related signaling molecules and JNK.

FIGS. 9a to 9m show that ROS induced JNK-mediated-EGFR turnover. Specifically, FIGS. 9a and b: EGFR-mutant NSCLCs were plated in complete media that was replaced the following day with glucose-free medium, incubated or treated with 2DG (10 mM) or rotenone (5 μM) for 24 hours, and subjected to DCFDA assay (FIG. 9a) or MitoSox Red assay (FIG. 9b); FIG. 9c: EGFR-mutant NSCLCs were treated with H₂O₂ at indicated doses for 24 hours and assayed for cell death by Annexin V/PI staining and flow cytometry; FIGS. 9d and 9e: EGFR-mutant NSCLCs were treated with H₂O₂ at indicated doses for 24 hours and immunoblotted with indicated antibodies; and FIGS. 9f to 9m: EGFR-mutant NSCLCs were plated in complete media that was replaced the following day with NAC-supplemented (4 mM) glucose-free medium, incubated or treated with either 2DG (10 mM) or rotenone (5 μM) for 24 hours in the absence or presence of NAC (4 mM), and assayed for cell death by Annexin V/PI staining and flow cytometry (FIGS. 9f and 9g), or immunoblotted with indicated antibodies (FIGS. 9h to 9m). NAC, N-acetyl-L-cysteine. *, P<0.05; **, P<0.01.

FIGS. 10a to 10c show that JNK is a downstream target of ROS. Specifically, EGFR-WT NSCLCs were treated with anisomycin for 24 h in the absence or presence of NAC and assayed for cell death by annexin V/PI staining and flow cytometry (FIG. 9a), or immunoblotted with indicated antibodies (FIGS. 9b and 9c).

FIGS. 11a to 11h show that autophagy induces EGFR degradation. Specifically, FIG. 11a: EGFR-mutant NSCLCs were plated in complete media that was replaced the following day with glucose-free medium or treated with either 2DG (10 mM) or anisomycin (5 μM) for 24 hours and immunoblotted with indicated antibodies; FIG. 11b: PC9 cells expressing GFP-LC3 were plated in complete media that was replaced the following day with glucose-free medium or treated with either 2DG (10 mM) or anisomycin (5 μM) for 24 hours and analyzed for LC3 dots; FIG. 11c: EGFR-mutant NSCLCs were plated in complete media that was replaced the following day with glucose-free medium or treated with 2DG (10 mM) for 24 hours in the absence or presence of SP600125, and immunoblotted with indicated antibodies; FIG. 11d: EGFR-mutant NSCLCs were treated with trehalose for 24 hours and immunoblotted with indicated antibodies; FIG. 11e: EGFR-mutant NSCLCs were plated in complete media that was replaced the following day with glucose-free medium or treated with 2DG (10 mM) for 24 hours in the absence or presence of chloroquine and immunoblotted with indicated antibodies; FIG. 11f: EGFR-mutant NSCLCs were treated with anisomycin (5 μM) for 24 hours in the absence or presence of chloroquine and immunoblotted with indicated antibodies; FIG. 11g: EGFR-mutant NSCLCs expressing a control (shGFP) or ATG7 shRNAs (shATG) were plated in the complete medium, which was replaced with glucose-free medium or treated with 2DG (10 mM) for 24 hours and immunoblotted with indicated antibodies; and FIG. 11h: EGFR-mutant NSCLCs expressing a control (shGFP) or ATG7 shRNAs (shATG) were treated with anisomycin (5 μM) for 24 hours and immunoblotted with indicated antibodies. TRE, trehalose, CQ, chloroquine.

FIGS. 12a to 12d show that activated autophagy induces apoptotic cell death in EGFR-mutant NSCLCs. Specifically, FIG. 12a: EGFR-mutant NSCLCs were plated in the complete medium. On the following day, either this medium was replaced by glucose-free medium or the NSCLCs were treated with 2DG (10 mM) for 24 h in the absence or presence of chloroquine (10 μM), followed by immunoblotting with indicated antibodies; FIG. 12b: EGFR-mutant NSCLCs were treated with anisomycin (5 μM) for 24 h in the absence or presence of chloroquine and immunoblotted with indicated antibodies; FIG. 12c: EGFR-mutant NSCLCs expressing a control (shGFP) or ATG7shRNAs (shATGs) were plated in the complete medium, which was replaced with glucose-free medium or treated with 2DG (10 mM) for 24 h in the absence or presence of chloroquine and immunoblotted with indicated antibodies; and FIG. 12d: EGFR-mutant NSCLCs expressing a control (shGFP) or ATG7 shRNAs (shATGs) were treated with anisomycin (5 μM) for 24 h in the absence or presence of chloroquine and immunoblotted with indicated antibodies.

FIGS. 13a to 13j show that JNK activation sensitizes EGFR-TKI-resistant NSCLCs to apoptosis. Specifically, FIGS. 13a and 13b: Cells were plated in complete media that was replaced the following day with glucose or glutamine-free medium, incubated for another 24 hours, and assayed for cell growth; FIGS. 13c and 13d: Cells were plated in complete media that was replaced the following day with glucose or glutamine-free medium, incubated for another 24 hours, and assayed for cell death by Annexin V/PI staining and flow cytometry; FIGS. 13e and 13f: Cells were treated with 2DG (10 mM) or BPTES (10 μM) for 24 hours and assayed for cell death by Annexin V/PI staining and flow cytometry; FIG. 13g: Cells were plated in complete media that was replaced the following day with glucose or glutamine-free medium, or treated with either 2DG (10 mM) or BPTES (10 μM) for 24 hours, and immunoblotted with indicated antibodies; FIG. 13h: Cells were treated with anisomycin for 24 hours and immunoblotted with indicated antibodies; and FIGS. 13i and 13j: Cells were treated with anisomycin for 24 hours and assayed for cell death by Annexin V/PI staining and flow cytometry. *, P<0.05; **, P<0.01.

FIGS. 14a to 14d show that glucose deprivation has minimal effect on the growth of EGFRTKIs-resistant cell lines without EGFR dependency. Specifically, FIG. 14a: Western blotting confirmed EGFR knockdown; FIG. 14b: EGFR-TKI-resistant cell lines expressing control shRNA (shGFP) or two independent shRNAs to EGFR were assayed for cell viability via MTT assay; and FIGS. 14c and 14d: Cells were plated in complete media that was replaced the following day with glucose or glutamine-free medium, incubated for another 24 h, and assayed for cell growth.

FIGS. 15a to 15g show immunohistochemical staining of P-JNK and EGFR in TMA blocks. Specifically, FIGS. 15a and 15d show photographs of P-JNK (FIG. 15a) and EGFR (FIG. 15d) staining, FIGS. 15b and 15c show that the phosphorylated JNK expression is significantly reduced in TMA tissues with EGFR mutation, FIGS. 15e and 15f show a significant negative correlation between P-JNK and EGFR expression, and FIG. 15g is a model depicting EGFR-regulated aerobic glycolysis in EGFR-mutant NSCLCs used to inhibit autophagy-mediated EGFR degradation.

### DETAILED DESCRIPTION OF THE INVENTION

The present invention will now be described in detail.

The present invention provides a pharmaceutical composition and method for preventing or treating an EGFR-mutant non-small cell lung cancer including a c-Jun N-terminal kinase (JNK) activator as an active ingredient.

The JNK activator is not particularly limited but is preferably anisomycin or a derivative thereof. Anisomycin has the chemical name (2S,3S,4S)-4-hydroxy-[(4-methoxyphenyl)methyl]-pyrrolidin-3-yl-acetate.

As used herein, the term “non-small cell lung cancer” has its general meaning in the art. For example, non-small cell lung cancer cells are malignant cells arising from the epithelial cells of the lung. Non-small cell lung cancers are categorized by the size and appearance of the tumor cells observed by a histopathologist under a microscope.

According to one embodiment of the present invention, the non-small cell lung cancer is squamous cell carcinoma, adenocarcinoma, large cell carcinoma, adenosquamous carcinoma or sarcomatoid carcinoma.

According to one embodiment of the present invention, the non-small cell lung cancer harbors an EGFR mutation. The term “EGFR mutation” refers to a variance in the nucleotide sequence of ErbB1, the gene encoding the EFFR, that results in an increase kinase activity. The increased kinase activity is a direct result of the variance in the nucleic acid and is associated with the protein for which the gene encodes.

In one particular embodiment, the EGFR mutation is an in-frame deletion mutation in exon 19 or a point mutation in exon 21. Examples of in-frame deletion mutations in exon 19 include mutations in E746-A750del, S752-1759del, K747-A750del, and K747-E749+A750. Examples of point mutations in exon 21 include L858R and L861Q point mutations.

According to one embodiment of the present invention, the non-small cell lung cancer further harbors an EGFR mutation causing resistance to a reversible EGFR TKI in addition to the EGFR-activating mutation. In one particular embodiment, the EGFR mutation causing resistance to a reversible EGFR TKI is a T790 M mutation in exon 20 of EGFR.

According to one embodiment of the present invention, the non-small cell lung cancer may be resistant to a reversible EGFR-TKI such as gefitinib or erlotinib.

Reversible EGFR-TKIs are effective in treating non-small cell lung cancers harboring EGFR-activating mutations such as E746-A750 deletion mutation and L858R point mutation but are no longer effective in treating non-small cell lung cancers harboring secondary mutations such as T790 M because of their resistance to reversible EGFR-TKIs. Irreversible second-generation EGFR-TKIs (such as afatinib) have been developed to treat non-small cell lung cancers resistant to reversible EGFR-TKIs but they exhibit limited effects on non-small cell lung cancers with acquired resistance and are thus unsatisfactory in the treatment of non-small cell lung cancers.

The pharmaceutical composition and method of the present invention is also effective in treating and preventing non-small cell lung cancers resistant to EGFR-TKIs due to the presence of the c-Jun N-terminal kinase (JNK) activator.

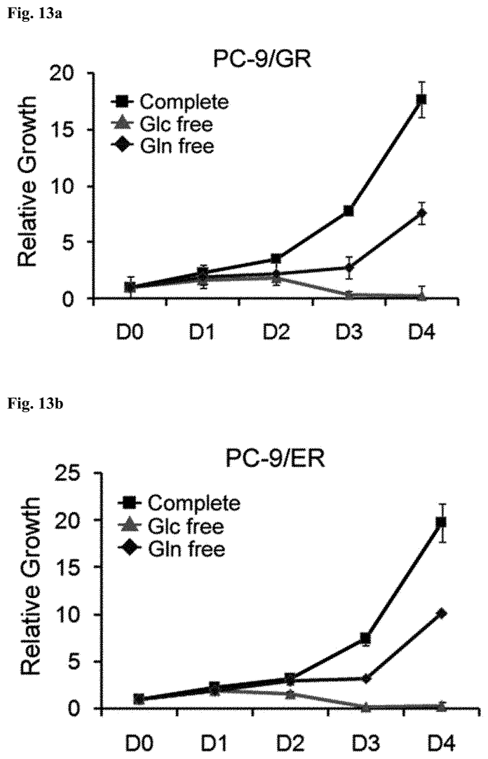

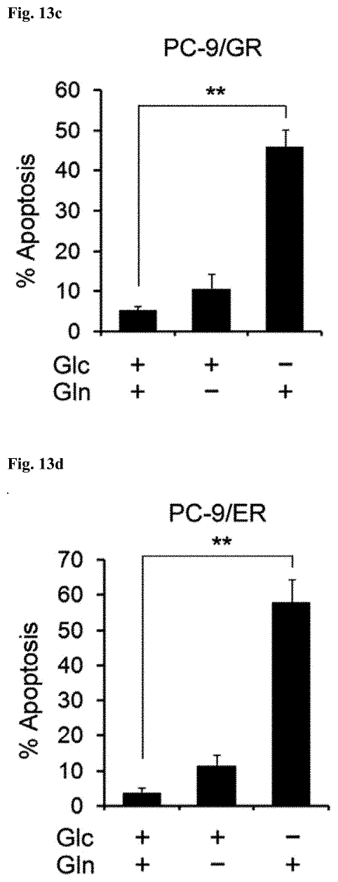

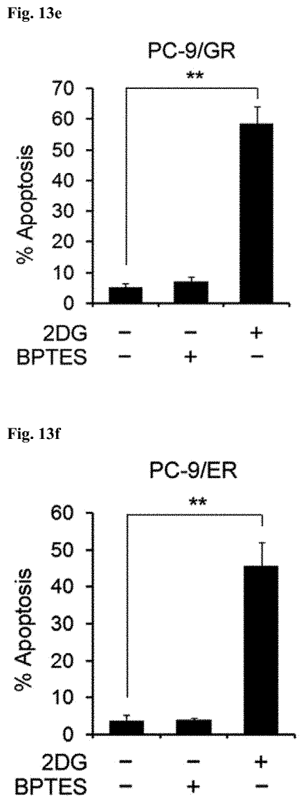

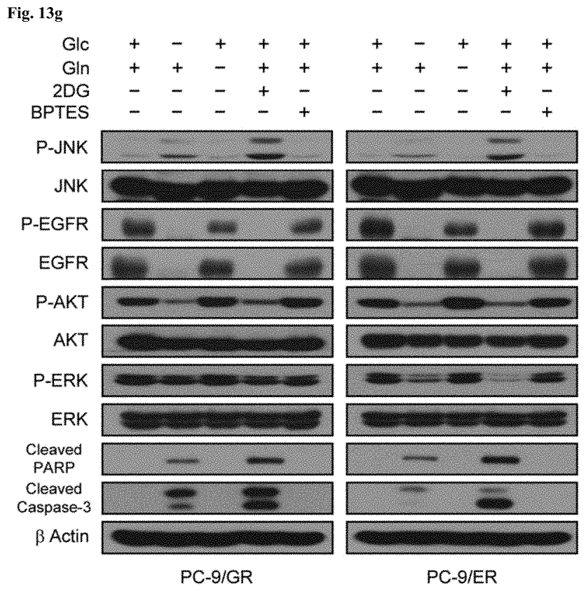

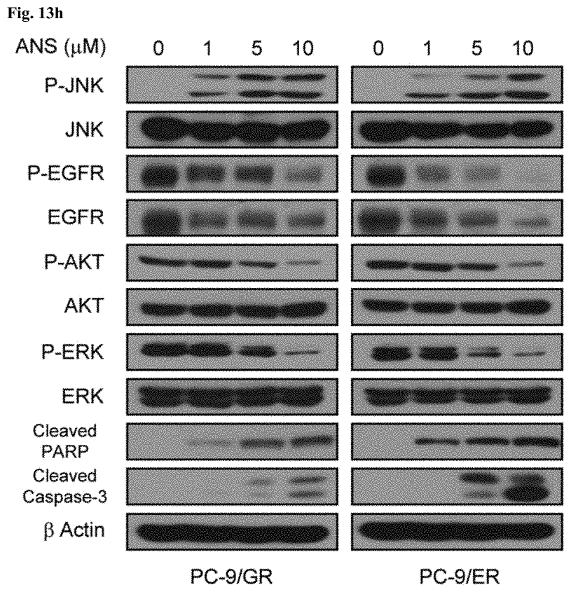

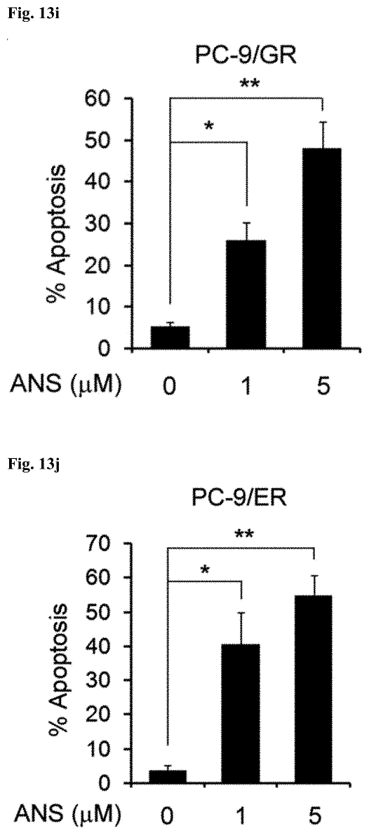

The c-Jun N-terminal kinase (JNK) activator inhibits the growth of non-small cell lung cancer cells harboring a T790 M mutation (i.e. resistant to EGFR-TKIs) and significantly induces apoptosis, as demonstrated in the Examples section that follows (see FIGS. 13i and 13j).

The c-Jun N-terminal kinase (JNK) activator can selectively inhibit EGFR mutants without inhibiting wild-type EGFR.

The pharmaceutical composition may further include a pharmaceutically acceptable carrier, excipient or diluent known in the art.

Examples of carriers, excipients or diluents suitable for use in the pharmaceutical composition of the present invention include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

The pharmaceutical composition of the present invention can be formulated into oral preparations, such as powders, granules, tablets, capsules, suspensions, emulsions, syrups, and aerosols, and other preparations, such as external preparations, suppositories, and sterile injectable solutions, according to a conventional method suitable for each preparation.

The pharmaceutical composition of the present invention may be formulated with diluents or excipients commonly used in the art, such as fillers, extenders, binders, wetting agents, disintegrating agents or surfactants. Examples of solid preparations for oral administration include tablets, pills, powders, granules, and capsules. Such solid preparations may be prepared by mixing the pharmaceutical composition with at least one excipient, for example, starch, calcium carbonate, sucrose, lactose or gelatin.

The pharmaceutical composition of the present invention may use one or more lubricating agents such as magnesium stearate and talc, in addition to simple excipients. The pharmaceutical composition of the present invention can be formulated into liquid preparations for oral administration, such as suspensions, liquids for internal use, syrups, and emulsions. Such liquid preparations may include simple diluents commonly used in the art, for example, water and liquid paraffin, and various types of excipients, for example, wetting agents, sweetening agents, flavoring agents, and preservatives.

The pharmaceutical composition of the present invention can be formulated into preparations for parenteral administration. Examples of such preparations for parenteral administration include sterile aqueous solutions, non-aqueous solvents, suspensions, emulsions, freeze-drying agents, and suppositories. The non-aqueous solvents and the suspensions may be propylene glycol, polyethylene glycol, vegetable oils such as olive oil, and injectable esters such as ethyl oleate. Witepsol, macrogol, Tween 61, cacao butter, laurin butter, and glycerogelatin may be used as bases of the suppositories.

The c-Jun N-terminal kinase (JNK) activator may be present in an amount of 0.01 to 40% by weight, preferably 0.1 to 30% by weight, based on the total weight of the pharmaceutical composition. If the content of the JNK activator is less than the lower limit, its effects to inhibit the EGFR T790 M mutant, to overcome resistance to EGFR-TKIs, and to induce apoptosis in EGFR-mutant NSCLCs are negligible. Meanwhile, if the content of the JNK activator exceeds the upper limit, the effect of adding the JNK activator is negligible.

The amount of the c-Jun N-terminal kinase (JNK) activator used in the pharmaceutical composition of the present invention may vary depending on the age, sex, and body weight of patients and the severity of the disease. The pharmaceutical composition is administered typically in an amount of 0.001 to 100 mg/kg, preferably 0.01 to 10 mg/kg, one or more times daily.

The dose of the c-Jun N-terminal kinase (JNK) activator may be appropriately increased or reduced taking into consideration the route of administration, the severity of the disease, and the sex, body weight and age of patients. Accordingly, the dose is not in no way intended to limit the scope of the invention.

The pharmaceutical composition of the present invention can be administered to mammals, including rats, mice, livestock, and humans, via various routes. All routes of administration may be contemplated. The pharmaceutical composition of the present invention may be administered by any suitable route, for example, orally, rectally, intravenously, intramuscularly, subcutaneously, intrabronchially, intrauterinely or intracerebroventricularly.

In a further aspect, the present invention provides a composition and method for inhibiting the resistance of a non-small cell lung cancer to an EGFR tyrosine kinase inhibitor (hereinafter referred to as ‘TKI’) including a c-Jun N-terminal kinase (JNK) activator as an active ingredient.

The active ingredient of the composition is the same as that of the pharmaceutical composition and a description thereof is omitted to avoid excessive complexity of the specification.

As used herein, the term “resistance” refers to the resistance of non-small cell lung cancer cells to anticancer agents such as reversible EGFR-TKIs. This term means that EGFR-TKIs such as gefitinib have no therapeutic effect on lung cancer patients from the initial stage of treatment or lose their therapeutic effect during continued treatment despite their therapeutic effect on cancer at the initial stage of treatment. As such, continuous administration of an anticancer agent to a cancer patient is known to lead to a gradual reduction in the effect of the anticancer agent in the cancer patient. Particularly, non-small cell lung cancers are known to acquire resistance to reversible EGFR-TKIs by EGFR mutation.

According to one embodiment of the present invention, the non-small cell lung cancer is resistant to an EGFR-TKI due to a T790 M mutation in exon 20 of EGFR. In one particular embodiment, the EGFR-TKI is gefitinib or erlotinib.

The c-Jun N-terminal kinase (JNK) activator overcomes resistance of the non-small cell lung cancer to the EGFR-TKI. Therefore, the composition of the present invention is expected to be useful for the treatment of non-small cell lung cancer patients with little or no response to reversible EGFR-TKIs.

In another aspect, the present invention provides a health functional food composition for preventing or ameliorating an EGFR-mutant non-small cell lung cancer including a c-Jun N-terminal kinase (JNK) activator as an active ingredient.

The active ingredient of the health functional food is the same as that of the pharmaceutical composition and a description thereof is omitted to avoid excessive complexity of the specification.

As used herein, the term “ameliorating” refers to all actions that at least reduce a parameter related to the conditions to be treated, for example, the degree of symptom.

The health functional food composition of the present invention can be used as a food additive. In this case, the health functional food composition may be added without further processing or may be optionally used in combination with one or more other foods or food ingredients in accordance with methods known in the art.

There is no particular restriction on the kind of the food. Non-limiting examples of foods that may be added with the health functional food composition include all common foods, such as meats, sausages, breads, chocolates, candies, snacks, crackers, cookies, pizza, flour products (e.g., instant noodles), chewing gums, dairy products (including ice creams), soups, beverages, teas, drinks, alcoholic drinks, and vitamin complexes.

The health functional food composition of the present invention may be a beverage composition. In this case, the health functional food composition of the present invention may further include various flavoring agents or natural carbohydrates, like general beverages. Non-limiting examples of the natural carbohydrates include monosaccharides such as glucose and fructose, disaccharides such as maltose and sucrose, natural sweetening agents such as dextrin and cyclodextrin, and synthetic sweetening agents such as saccharin and aspartame. The proportions of the additional ingredients can be appropriately determined by those skilled in the art.

The health functional food composition of the present invention may further contain one or more additives selected from nutrients, vitamins, electrolytes, flavoring agents, coloring agents, pectic acid and its salts, alginic acid and its salts, organic acids, protective colloidal thickeners, pH adjusting agents, stabilizers, preservatives, glycerin, alcohols, and carbonating agents for carbonated drinks. The health functional food composition of the present invention may further contain flesh for the production of natural fruit juices, fruit juice beverages, and vegetable beverages. Such ingredients may be used independently or as a mixture thereof. The proportions of such additives can also be appropriately determined by those skilled in the art.

The active ingredient may be present in an amount of 0.01 to 99% by weight, based on the total weight of the composition, but is not necessarily limited thereto. The content of the active ingredient may vary depending on the condition of patients and the type and severity of the disease.

According to one embodiment of the present invention, a subject in need thereof is administered a composition comprising a c-Jun N-terminal kinase (JNK) activator as an active ingredient in order to treat, ameliorate, or prevent an EGFR-mutant non-small cell lung cancer. In some embodiments, the composition can be formulated as described above; that is, e.g., a health functional food composition or a pharmaceutical composition. In some embodiments, the subject has been diagnosed with an EGFR-mutant non-small cell lung cancer. In some embodiments, a therapeutically effective amount of the composition is administered.

In some embodiments, the non-small cell lung cancer harbors a deletion mutation in exon 19 of EGFR or a point mutation in exon 21 of EGFR. In some embodiments, the non-small cell lung cancer harbors a T790 M mutation in exon 20 of EGFR. In some embodiments, the non-small cell lung cancer is resistant to gefitinib or erlotinib. In some embodiments, the non-small cell lung cancer is resistant to a reversible EGFR tyrosine kinase inhibitor due to a T790 M mutation in exon 20 of EGFR. In some embodiments, the non-small cell lung cancer is squamous cell carcinoma, adenocarcinoma, large cell carcinoma, adenosquamous carcinoma or sarcomatoid carcinoma.

According to one embodiment of the present invention, a subject in need thereof is administered a composition comprising a c-Jun N-terminal kinase (JNK) activator as an active ingredient in order to inhibit or decrease the resistance of a non-small cell lung cancer to an EGFR tyrosine kinase inhibitor in the subject. In some embodiments, the composition can be formulated as described above; that is, e.g., a health functional food composition or a pharmaceutical composition. In some embodiments, the subject has been diagnosed with an EGFR-mutant non-small cell lung cancer. In some embodiments, a therapeutically effective amount of the composition is administered.

In some embodiments, the JNK activator is anisomycin or a derivative thereof. In some embodiments, the JNK activator is present in an amount of 0.01 to 40% by weight, based on the total weight of the pharmaceutical composition. In some embodiments, the pharmaceutical composition is administered in an amount of 0.001 to 100 mg/kg/day. In some embodiments, the pharmaceutical composition further comprises a pharmaceutically acceptable carrier, excipient or diluent. In some embodiments, the pharmaceutical composition is formulated into a liquid, powder, aerosol, injectable preparation, Ringer's solution, patch, capsule, pill, tablet, depot or suppository.

The composition and method for preventing, ameliorating or treating an EGFR-mutant non-small cell lung cancer according to the present invention will be explained in detail with reference to the following examples.

### EXAMPLES

Materials and Methods

Cell Culture

EGFR WT (A549 and H1299) and EGFR-mutant NSCLC cells (HCC827 and H1975) were purchased from the American Type Culture Collection. The PC-9 cell line was a kind gift from Dr. Kazuto Nishio (National Cancer Center Hospital, Tokyo, Japan) and has been previously characterized. PC-9/GR (gefitinib-resistant cell line) and PC-9/ER (erlotinib-resistant cell line) cells were established as part of a previous study. All cells were maintained at 37° C. in humidified air with 5% CO₂ and in RPMI1640 medium supplemented with 10% FBS, 100 U/mL penicillin, and 100 μg/mL streptomycin (all from Thermo Scientific, Waltham, Mass., USA). The cell lines were used within 10 to 15 passages from the initial expansion and freeze-down, routinely tested for Mycoplasma contamination, and authenticated by short tandem repeat (STR) DNA profiling as described previously before the study. For glucose or glutamine deprivation, RPMI1640 without glucose (R1383) or without glutamine (R0883) was obtained from Sigma-Aldrich (St Louis, Mo., USA).

Cell Proliferation Assay

Cells were plated in 24-well plates (density: 2,000 cells/well). For nutrient deprivation, cells were plated in complete culture media (10 mM glucose, 2 mM glutamine), which was exchanged with glucose or glutamine-free medium the following day. Media was not changed throughout the course of the experiment. At the indicated time intervals, cells were fixed in 10% formalin and stained with 0.1% crystal violet. The dye was extracted with 10% acetic acid, and relative proliferation was determined according to the optical density (OD) at 595 nm.

Reagents and Antibodies

Anisomycin (1290) and SP600125 (1496) were obtained from Tocris Bioscience (Bristol, United Kingdom), and methyl-pyruvate (371173), rotenone (R8875), dimethyl α-KG (349631), BPTES (SML0601), and 2DG (2-deoxy-D-glucose; D6134) were purchased from Sigma-Aldrich. Antibodies to AKT (9272), p-AKT (4060), cleaved PARP (9541), cleaved-caspase-3 (9661), ERK (9102), and p-JNK (4668) were purchased from Cell Signaling Technology (Beverly, Mass., USA), antibodies to β-actin (sc-47778), EGFR (sc-03), p-EGFR (sc-12351), p-ERK (sc-7383), and JNK (sc-7345) were obtained from Santa Cruz Biotechnology (Dallas, Tex., USA).

Glucose Consumption and Lactate Production Assay

Cells were plated in 6-well plates (2×10⁵ cells/well). Media was not changed throughout the experimental course and was collected at the indicated time intervals. Glucose and lactate concentrations in media were measured using the YSI 2300 STAT Plus GlucoseLactate Analyzer.

ECAR and OCR Measurement

Extracellular acidification rate (ECAR) and oxygen consumption rate (OCR) were measured with an XF24 extracellular flux analyzer (Seahorse Bioscience). Briefly, cells were plated in a 24-well Seahorse plate and cultured at 37° C. with 5% CO₂, medium was replaced the following day with unbuffered DMEM, and cells were incubated at 37° C. without CO₂ for 1 hour. For OCR measurement, oligomycin, FCCP, and rotenone were added to final concentrations of 2 μM, 5 μM, and 2 μM, respectively. For ECAR measurements, glucose, oligomycin, and 2DG were added to final concentrations of 10 mM, 1 μM, and 20 μM, respectively.

Metabolomics

Cells were grown to ˜60% confluence in growth media (RPMI1640, 10% FBS) on 10-cm dishes. After 24 hours, cells were harvested using 1.4 mL of cold methanol/H₂O (80/20, v/v) after sequential PBS and H₂O washes, and lysed; 100 μL of 5 μM internal standard was added. Metabolites were liquid-liquid extracted from the aqueous phase after adding chloroform. The aqueous phase was dried via vacuum centrifugation, and the sample was reconstituted with 50 μL of 50% methanol prior to LC/MS-MS analysis. The LC/MS-MS system was equipped with an Agilent 1290 HPLC (Agilent Technologies, Santa Clara, Calif., USA), Qtrap 5500 (ABSciex, Concord, Ontario, Canada), and reverse phase column (Synergi fusion RP 50×2 mm). A 3 μL volume was injected into the LC/MS-MS system and ionized with a turbo spray ionization source. Multiple reaction monitoring was used in negative ion mode, and the extracted ion chromatogram (EIC), corresponding to the specific transition for each metabolite was used for quantitation. The area under the curve of each EIC was normalized to that of the internal standard EIC. The peak area ratio of each metabolite to the internal standard was normalized using protein amount in a sample, and then was used for relative comparison.

qRT-PCR

Total RNA was extracted using TRIzol (Invitrogen, Carlsbad, Calif., USA). cDNA was synthesized from 2 μg of total RNA, using oligo-dT and MMLV HP reverse transcriptase (Epicentre, Madison, Wis., USA) according to the manufacturer's instruction. qRT-PCR was performed on an AriaMax Real-Time PCR instrument (Agilent Technologies, Santa Clara, Calif., USA) using the SYBR detection protocol. cDNA was calculated by the comparative Ct method, with 18S ribosomal RNA as a control. PCR reactions were performed in triplicates.

ROS Quantification

To determine cytoplasmic ROS, cells were incubated with 2′,7′-dichlorodihydrofluorescein diacetate (DCFDA, 5 μM; Invitrogen, Carlsbad, Calif., USA) for 30 minutes. Excess DCFDA was removed by washing cells twice with PBS at room temperature; labeled cells were trypsinized, rinsed, and resuspended in PBS. The oxidation of DCFDA to the highly fluorescent 2′,7′-dichloro-fluorescein (DCF), which is proportionate to ROS generation, was analyzed by flow cytometry. To analyze mitochondrial ROS, the cells were then incubated with 5 μM MitoSOX™ reagent (Thermo Scientific, Waltham, Mass., USA) for 10 minutes at 37° C. and trypsinized, washed with PBS, and then resuspended in 200 μL of PBS. Stained cells were then quantified and analyzed on a flow cytometer (BeckmanCoulter, Brea, Calif., USA). Excitation wavelength was 510 nm, and emission wavelength was 580 nm.

Apoptosis Quantitation

Apoptotic cell death was detected using an Annexin-V/FITC assay. Cells were harvested by trypsinization, washed with PBS, and resuspended in Annexin-V binding buffer (10 mM HEPES, pH 7.4, 140 mM NaCl, 2.5 mM CaCl₂) containing Annexin-V FITC and propidium iodide. Stained cells were quantified and analyzed on a flow cytometer (BeckmanCoulter, Brea, Calif., USA).

Western Blot Analysis

Cells were lysed in RIPA lysis buffer containing protease inhibitor cocktail (Thermo Scientific, Waltham, Mass., USA); lysate concentrations were assayed using a BCA assay (Thermo Scientific). Equal amounts of lysates were mixed with Laemmli loading dye and boiled for 10 minutes. Lysates were subjected to SDS-PAGE, and separated proteins were transferred to PVDF membranes (EMD Millipore, Billerica, Mass., USA). Membranes were blocked in Tris-buffered saline (TBS) containing 5% nonfat dry milk and 0.1% Tween 20 (TBS-T) prior to overnight incubation with primary antibody at 4° C. After washing with TBS-T, blots were exposed to appropriate horseradish peroxidase-conjugated secondary antibodies for 1 hour. Proteins-antibody complexes were visualized on Kodak X-ray film using an enhanced chemiluminescence (ECL) detection system (Thermo Scientific, Waltham, Mass., USA).

Quantitation of Intracellular ATP

Intracellular ATP concentrations were measured using an ATP Colorimetric/Fluorometric Assay Kit (Biovision Incorporated, Milpitas, Calif., USA) according to the manufacturer's instructions. Briefly, cells were lysed in 100 mL of ATP assay buffer; 50 μL of supernatants were collected and added to a 96-well plate. To each well, 50 μL of ATP assay buffer containing ATP probe, ATP converter, and developer were added. Absorbance was measured at 570 nm.

Xenograft Studies

Female severe combined immunodeficiency (SCID) mice were purchased from Charles River Laboratories. All experimental procedures were approved by the Institutional Animal Care and Use Committee of Asan Institute for Life Sciences (protocol 2015-14-241). For each animal, 2×10⁶ cells mixed with Matrigel (BD Biosciences) were injected into the flank. Five mice per group were treated when the tumor volumes reached 50 to 100 mm³ with anisomycin (5 mg/kg, intratumoral, 5 days a week) or 2-deoxy-D-glucose (2DG) 500 mg/kg, intraperitoneal, 5 days a week) for 2 weeks. The length (L) and width (W) of each tumor were measured using calipers, and the tumor volume (TV) was calculated as TV=(L×W²)/2. To evaluate EGFR-related signaling in xenograft tumors, tumors were harvested after 3 days of drug treatment, lysed, and analyzed with Western blotting.

Lentiviral-Mediated shRNA Targets

The following RNAi Consortium clone IDs for shRNAs were used in this study: shEGFR-1 (TRCN0000195303) and shEGFR-2 (TRCN0000298822).

Tissue Microarrays

A tissue microarray (TMA) block was made from lung specimens of lung adenocarcinoma that were surgically resected between June 2009 and May 2016 at Asan Medical Center, Seoul, South Korea. These TMA block included 244 subjects containing 126 EGFR-mutant and 118 wild-type for EGFR mutation. The EGFR-mutant group included specimens with mutations in exon 19 or exon 21 only, except mutations in exon 18 or exon 20. The EGFR mutation status within exons 18 to 21 was analyzed by direct DNA sequencing using an automatic ABI PRISM 3100 Genetic Analyzer (Applied Biosystems, Foster City, Calif., USA) until August 2015, and thereafter the PNAClamp™ EGFR Mutation Detection Kit with PNA-mediated PCR clamping method. Medical records of study subject were retrospectively reviewed at April 2017. The study design was approved by the Institutional Review Board of Asan Medical Center, which waived the requirement for informed consent due to the retrospective nature of the analysis (project identification number 2016-0752). However, all study subjects had been provided informed consent for utilization of extracted lung for study after surgical resection.

Immunohistochemistry

Immunohistochemical (IHC) staining was done using a specific primary antibody including P-JNK (1:100; 700031; ThermoScientific, Rockford, Ill.) and EGFR (1:200; 28-0005; ThermoScientific). IHC data were made by pathologists at Asan Medical Center and Korea Cancer Center Hospital. Chi-square test was used to evaluate the differences between positive and negative expression of P-JNK and EGFR.

Statistical Analysis

All data were expressed as mean±standard deviation. Unpaired Student's t-test was used to determine the statistical significance between groups. P<0.05 was considered statistically significant.

Results

Oncogenic EGFR-Mediated Enhanced Glycolysis is Required for Maintaining EGFR Levels

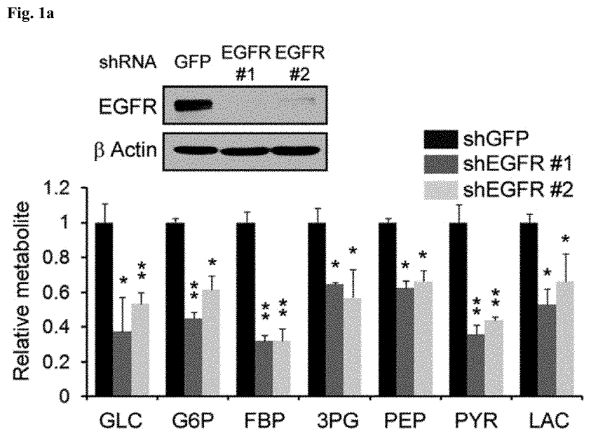

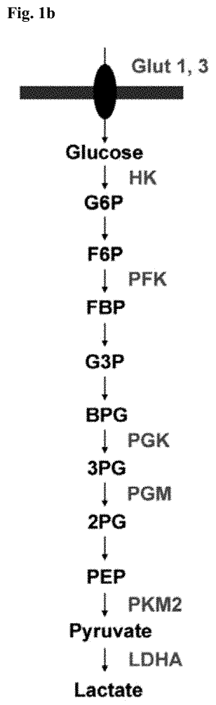

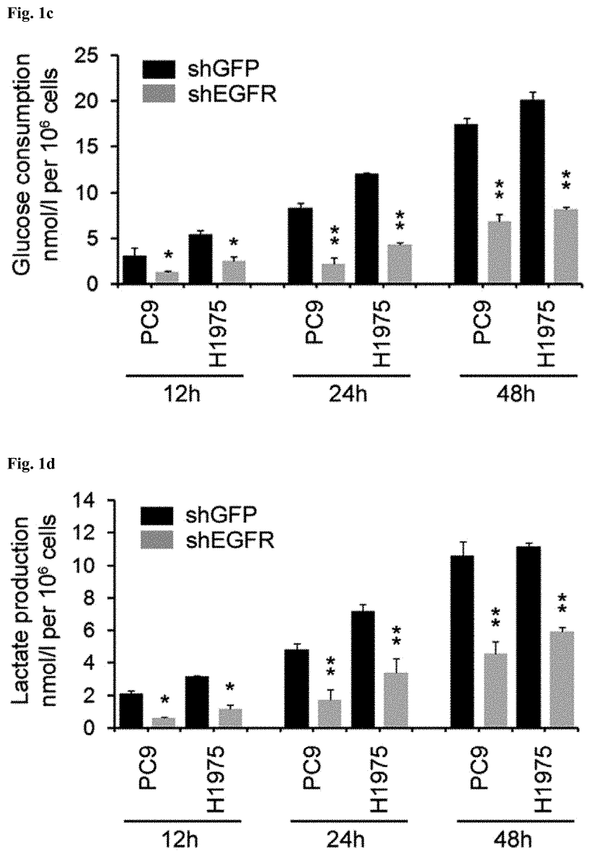

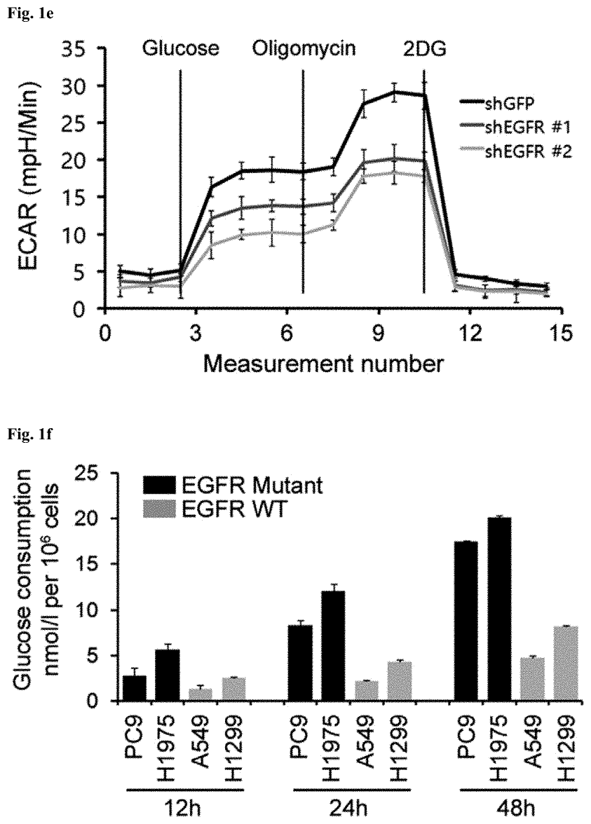

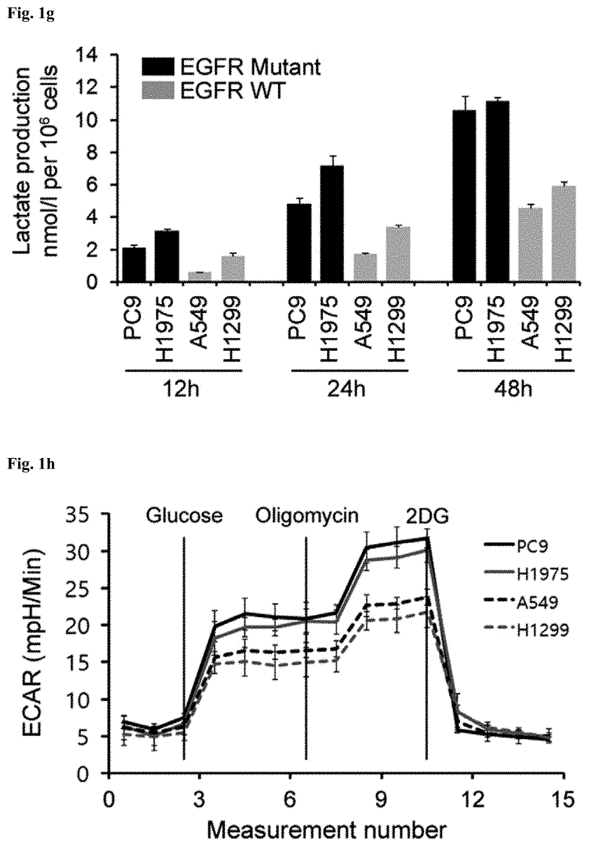

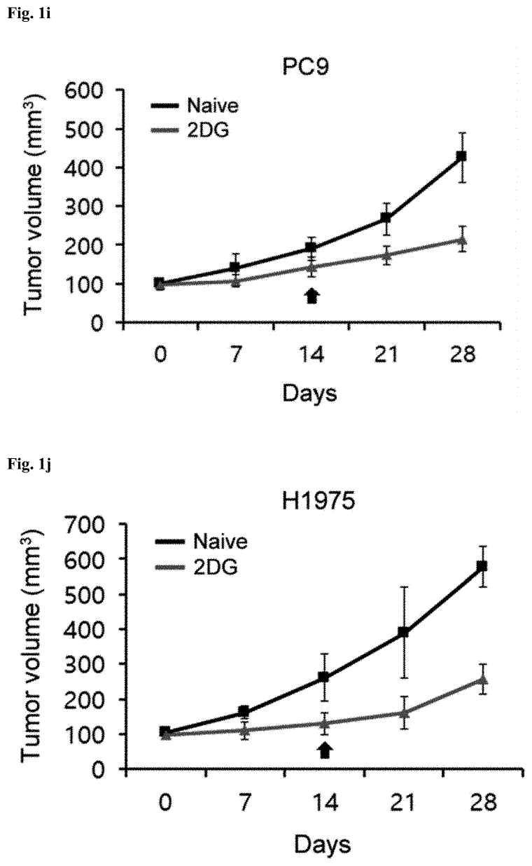

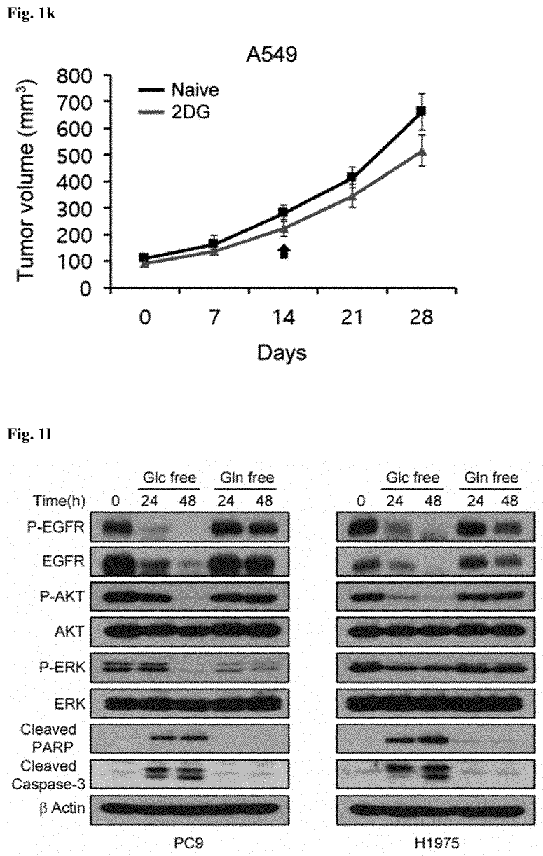

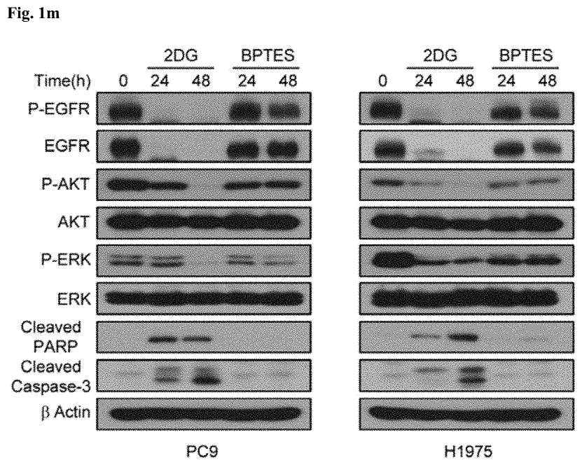

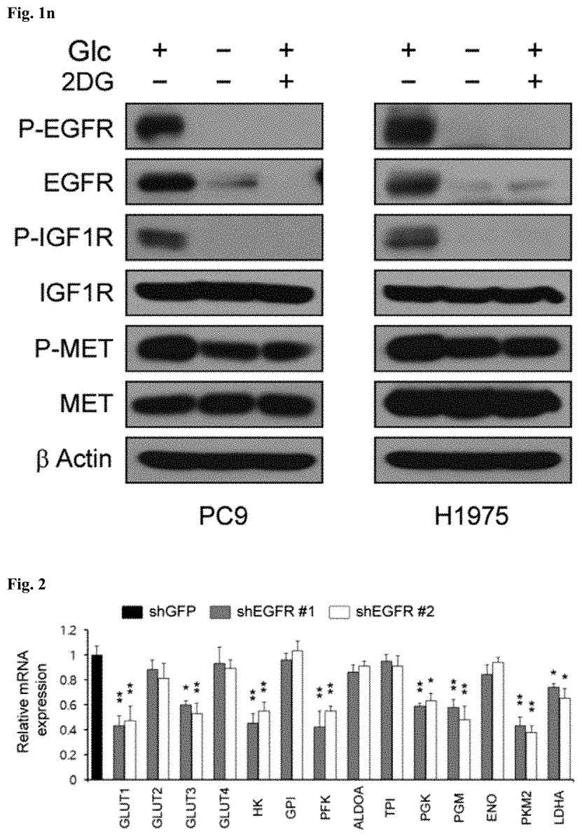

EGFR mutations are critical for clinical responses and prolonged survival of TKI-treated patients with non-small cell lung cancer (NSCLC), but the importance of EGFR mutation in lung cancer metabolism is unknown. To examine the functional role of EGFR mutation in NSCLC metabolism, the effect of EGFR on glucose metabolism was investigated via targeted liquid chromatography-tandem mass spectrometry (LC-MS/MS) metabolomic analysis of EGFR-mutant NSCLCs. Consistent with a recent study showing that EGFR-TKI treatment decreases glycolysis metabolites, EGFR knockdown was observed to lead to a significant decrease in glycolysis metabolites (FIG. 1a). Oncogenic signaling components such as Myc and HIF-1 mediate metabolic reprogramming via transcriptional regulation, and EGFR signaling pathway inhibition leads to decreased levels of GLUT1 and hexokinase 2 mRNA. Accordingly, EGFR knockdown reduced glycolytic gene expression at the transcription level (FIG. 1b and FIG. 2), and also reduced glucose uptake and lactate production in EGFR-mutant NSCLCs (FIGS. 1c and 1d). EGFR knockdown also decreased extracellular acidification rate (FIG. 1e), suggesting that EGFR mutation enhances glycolytic flux through transcriptional regulation.

To further define the function of EGFR in glycolysis regulation, glucose uptake and lactate production in EGFR-mutant NSCLCs were compared with those in EGFR-WT NSCLCs. As shown in FIGS. 1f and 1g, EGFR-mutant NSCLCs exhibited significantly elevated glucose uptake and lactate production compared with EGFR-WT NSCLCs, and the relative changes in glucose uptake and lactate production increased gradually over time. In addition, EGFR-mutant NSCLCs had a significantly higher extracellular acidification rate compared with EGFR-WT NSCLCs (FIG. 1h); this finding indicates that EGFR-mutant NSCLCs have a higher glycolytic rate.

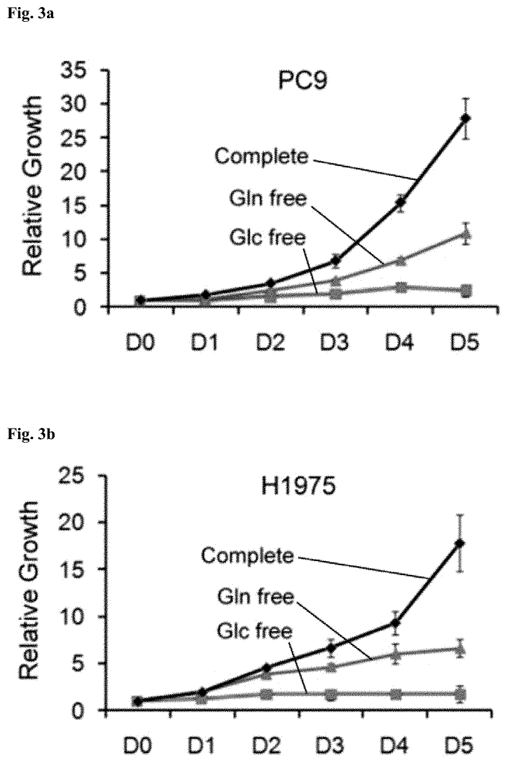

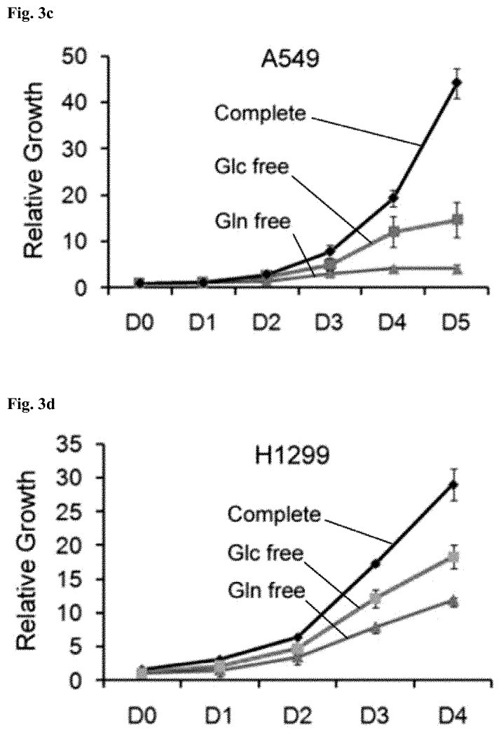

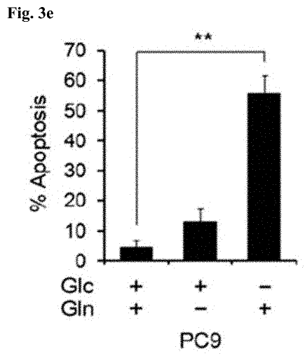

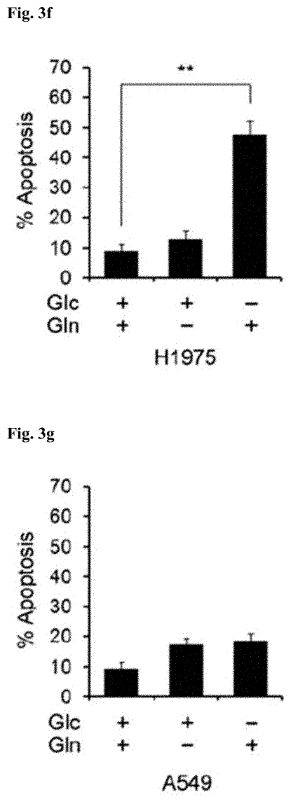

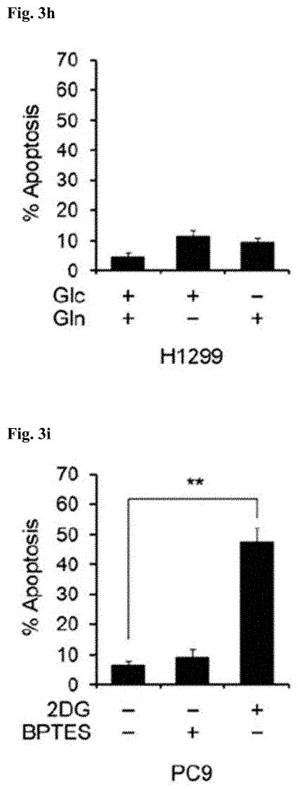

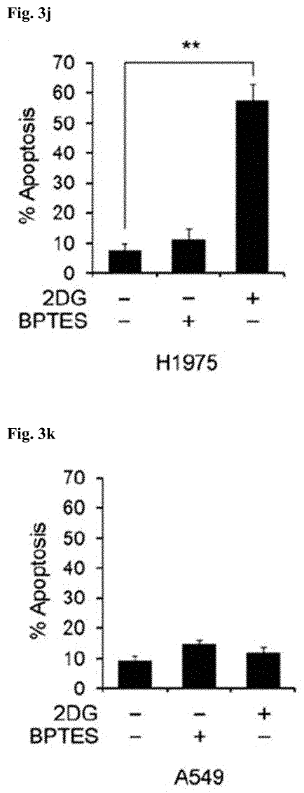

It was tested whether enhanced glycolysis is needed for growth support in EGFR-mutant NSCLC cells. Glucose deprivation markedly decreased EGFR-mutant NSCLC cell proliferation (FIGS. 3a and 3b), but had minimal effects on EGFR-WT NSCLCs (FIGS. 3c and 3d). To confirm the requirement of aerobic glycolysis for EGFR-mutant NSCLC survival, cell death in response to glucose deprivation was examined. As a result, significant apoptotic cell death was triggered in EGFR-mutant NSCLCs following glucose deprivation (FIGS. 3e and 3f), whereas glucose deprivation did not sensitize EGFR-WT NSCLCs to apoptosis (FIGS. 3g and 3h). Consistently, 2DG treatment, but not BPTES, promoted apoptosis only in EGFR-mutant NSCLCs (FIGS. 3i and 3l). For reference, 2DG is a glucose molecule whose 2-hydroxyl group is replaced by hydrogen and has the molecular formula C₆H₁₂O₅. 2DG is known to inhibit glycolysis. BPTES is an inhibitor of glutaminase 1 (GLS 1) and treatment of NSCLC cells with BPTES is known to significantly reduce ATP and NADH.

For further confirmation of the importance of aerobic glycolysis for the survival of EGFR-mutant NSCLCs, the ability of EGFR-mutant NSCLC cells to grow in vivo as a xenograft was assessed. As shown in FIGS. 1i, 1j, and 1k, 2DG treatment robustly diminished the growth of EGFR-mutant NSCLCs compared with that of EGFR-WT NSCLCs. These data suggest that EGFR mutation mediates glucose metabolism reprogramming via transcriptional regulation to promote tumor survival.

EGFR-mutant NSCLCs, which depend on EGFR for growth and survival, rely more strongly on EGFR signaling than do EGFR-WT NSCLCs. Given the importance of glucose metabolism in growth and survival, it was speculated that glucose metabolism might be needed for EGFR signaling in EGFR mutant NSCLCs. Therefore, it was tested whether nutrient starvation would affect EGFR signaling in this condition. Glutamine (Gln) deprivation, which had no effect on growth or survival, did not significantly affect EGFR signaling, whereas glucose deprivation markedly decreased EGFR levels in a time-dependent manner and inhibited phosphorylation of the EGFR signaling components AKT and ERK (FIG. 1l). Consistent with these results (FIGS. 3e and 3f), PARP and caspase-3 were cleaved only upon glucose deprivation (FIG. 1l). BPTES treatment had no effect on EGFR signaling, whereas 2DG treatment resulted in robust reduction of EGFR levels and inhibition of EGFR signaling in EGFR-mutant NSCLCs, thereby leading to apoptosis (FIG. 1m). Given that glucose metabolism inhibition led to a robust apoptotic cell death, it was speculated that glucose metabolism inhibition might suppress other receptor tyrosine kinases (RTK). Thus, phosphorylation and the total level of other RTKs upon glucose metabolism inhibition were examined. As shown in FIG. 1n, glucose deprivation or 2DG treatment significantly decreased IGF1R phosphorylation and marginally decreased MET phosphorylation; conversely, the total form of either did not change in response to glucose deprivation or 2DG treatment, indicating that the combined inhibition of activation of RTKs may activate profound apoptotic cell death. Taken together, these data demonstrate that EGFR mutation mediated enhanced glycolysis supports EGFR-mutant NSCLCs survival by maintaining EGFR levels.

Glucose as TCA Cycle Fuel is Essential for Survival of EGFR-Mutant NSCLCs

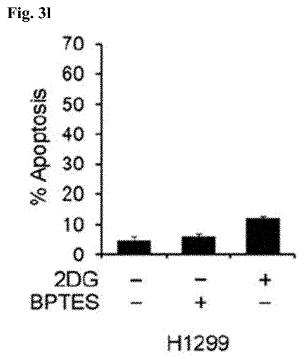

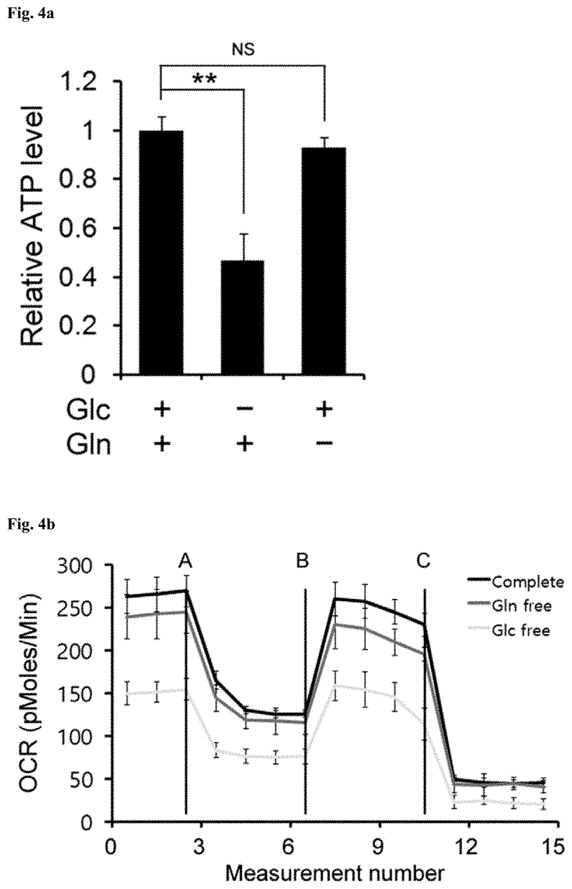

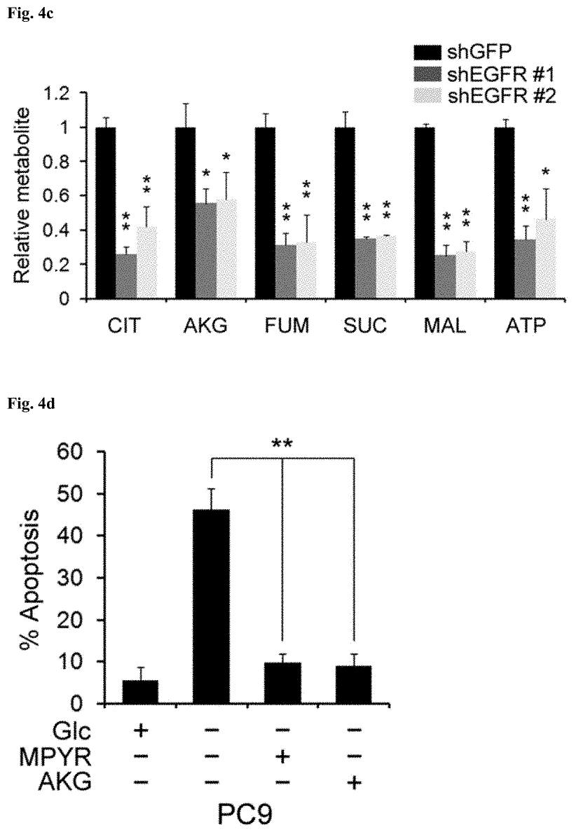

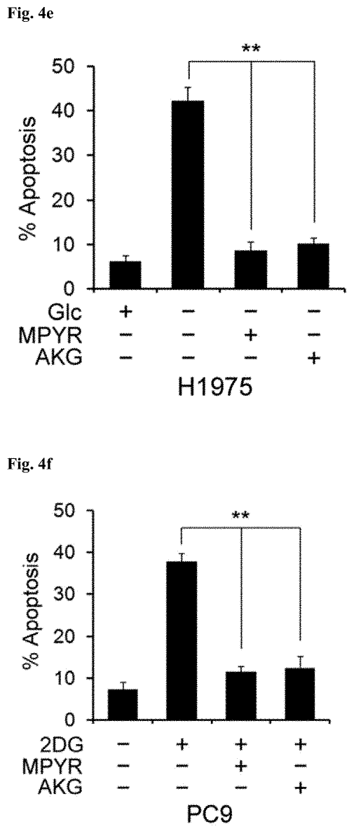

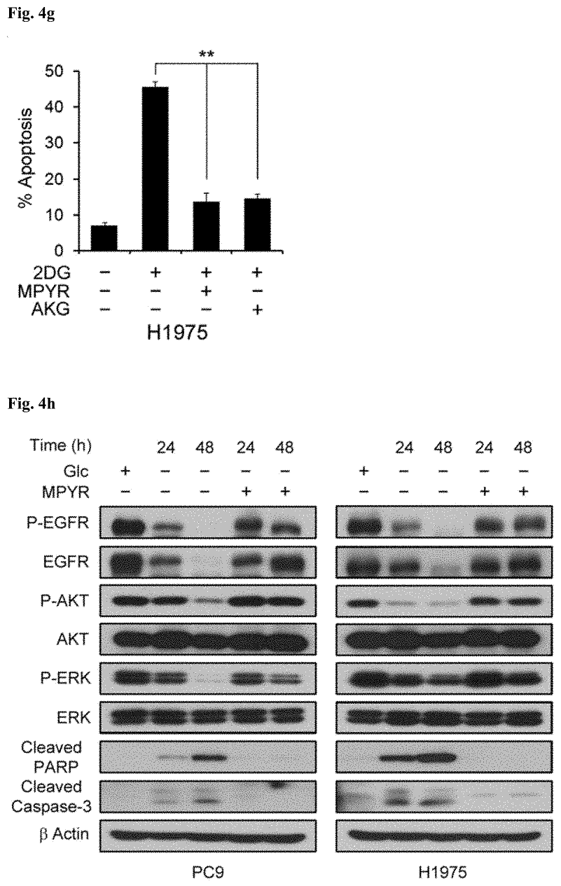

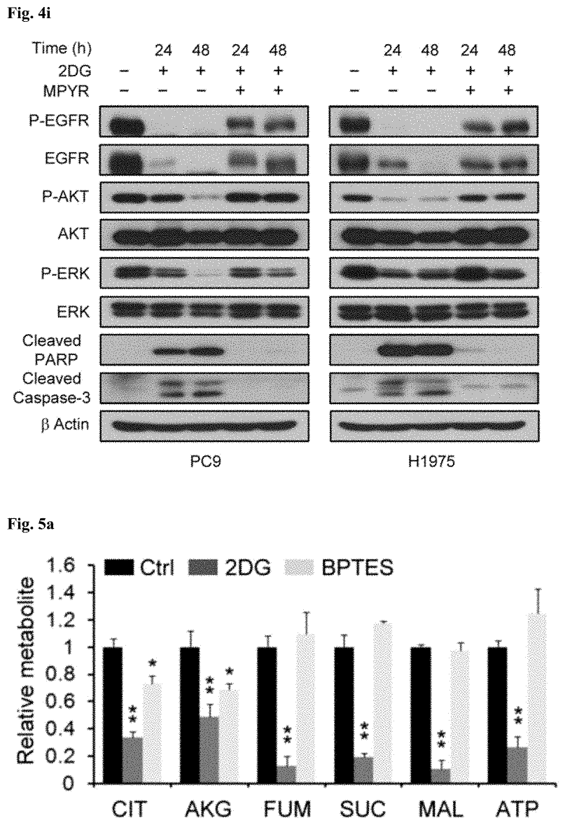

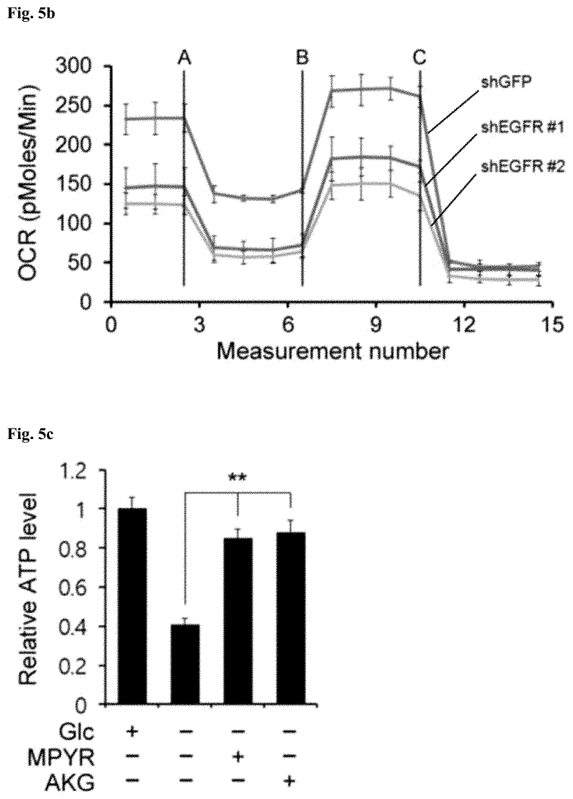

Glucose and glutamine are the major sources of energy and biosynthesis in proliferating tumor cells. Cells convert glucose for use in anabolic processes, whereas glutamine, an alternative energy source, is used to fuel the TCA cycle. Blocking glutamine metabolism as a source of TCA cycle fuel impairs tumor growth. Based on the previous observation that glucose, but not glutamine, deprivation significantly reduced growth and survival, it was suspected that unlike other tumors, EGFR-mutant NSCLCs might utilize glucose as a source of carbon fuel for the TCA cycle. Thus, ATP levels in EGFR-mutant NSCLCs in the absence of either glucose or glutamine were first examined. As a result, it was observed that ATP levels were not significantly affected by glutamine deprivation, but markedly reduced by glucose deprivation (FIG. 4a). To validate the essentiality of glucose for mitochondrial ATP production, oxygen consumption rates were measured. As shown in FIG. 4b, oxygen consumption decreased significantly upon glucose deprivation, compared with glutamine deprivation. To explore the direct effect of nutrient deprivation on levels of TCA cycle intermediates, a metabolic analysis was performed. BPTES treatment had no significant inhibitory effect on TCA cycle intermediates, whereas 2DG treatment significantly decreased the levels of TCA cycle intermediates (FIG. 5a). It was next examined whether EGFR is essential for mitochondrial metabolic function and energy level maintenance via increased glycolytic flux. EGFR knockdown led to significant decreases in the oxygen consumption rate as well as TCA intermediate levels (FIGS. 4c and 5b). These results suggest that EGFR-mediated enhanced glycolysis is a major source of carbon for TCA cycle in EGFR-mutant NSCLC.

To test whether the glucose metabolism inhibition-induced cell death results from insufficient maintenance of mitochondrial ATP production, an attempt was made to rescue glucose metabolism inhibition-induced cell death by supplementing the cells with either pyruvate or α-ketoglutarate, which provides substrates for the TCA cycle. Both pyruvate and α-ketoglutarate supplementation were observed to rescue EGFR-mutant NSCLCs from glucose deprivation-induced apoptosis (FIGS. 4d and 4e). Consistently, 2DG treatment-induced apoptotic cell death was reversed significantly upon pyruvate or α-ketoglutarate supplementation (FIGS. 4f and 4g). In addition, it was found that both pyruvate and α-ketoglutarate supplementation led to a significant recovery of ATP levels, which were reduced by glucose deprivation (FIG. 5c).

To further define the functional role of glucose metabolism in EGFR level maintenance, an attempt was made to see if the reduced EGFR level resulting from glucose metabolism inhibition can be restored by pyruvate supplementation. As shown in FIGS. 4h and 4i, pyruvate supplementation led to a significant recovery of EGFR levels reduced by the inhibition of glucose metabolism via glucose deprivation or 2DG treatment, and also restored the AKT and ERK phosphorylation. In the presence of pyruvate, the cleavage of PARP and caspase-3 upon glucose deprivation or 2DG treatment was inhibited as well. These results show that glucose, as TCA cycle fuel, is indispensable for sustaining EGFR levels in EGFR-mutant NSCLCs.

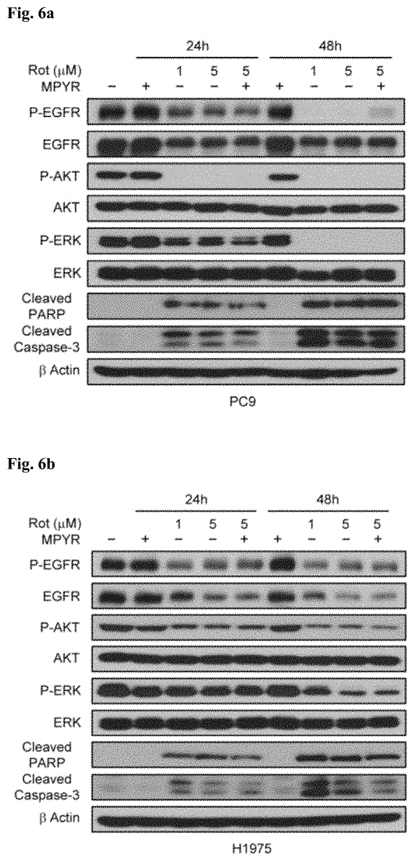

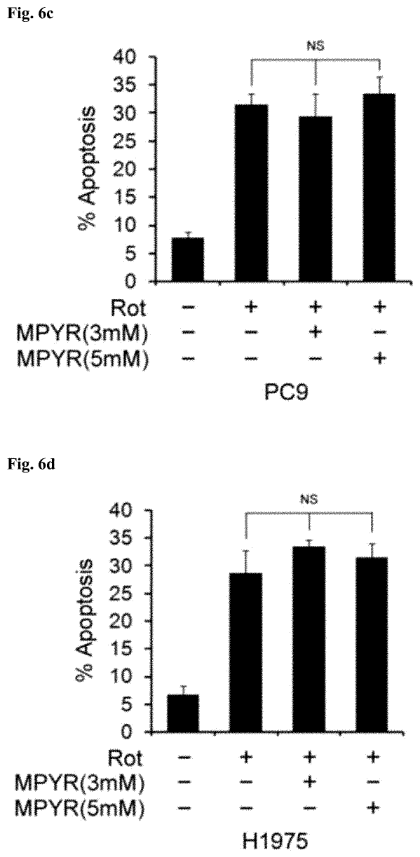

To confirm the importance of glucose-derived ATP production in mitochondria for EGFR level maintenance, EGFR levels were assessed following mitochondrial respiratory chain inhibition using the complex I inhibitor rotenone. Consistent with the results in FIGS. 6a to 6d, inhibition of mitochondrial ATP production with rotenone robustly decreased EGFR levels in both dose- and time-dependent manners (FIGS. 6a and 6b). Given the importance of the mitochondrial respiratory chain in EGFR level maintenance, it was speculated that excess pyruvate might not rescue the reduced EGFR levels following rotenone treatment, because the mitochondrial respiratory chain cannot operate under complex I inhibition. Indeed, it was observed that excess pyruvate could not rescue the decreased EGFR levels after rotenone treatment (FIGS. 6a and 6b). Similarly, pyruvate supplementation could not rescue EGFR-mutant NSCLCs from rotenone-mediated apoptosis (FIGS. 6c and 6d). Taken together, these data demonstrate that mitochondrial ATP production is critical for maintaining EGFR levels.

JNK Activation Inhibits EGFR-Mutant NSCLC Cell Survival Via Reduced EGFR Levels

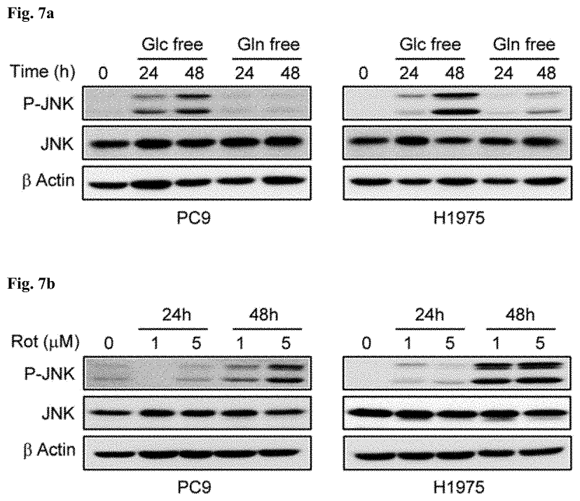

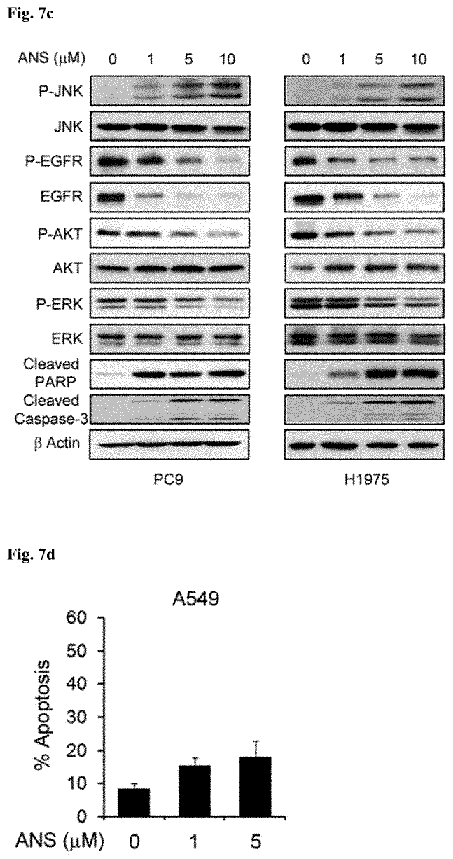

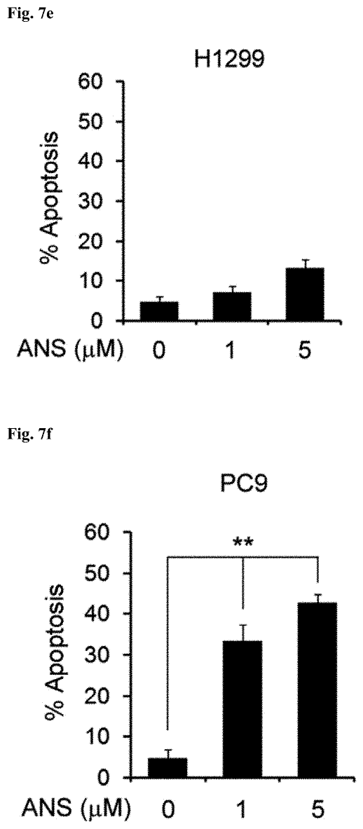

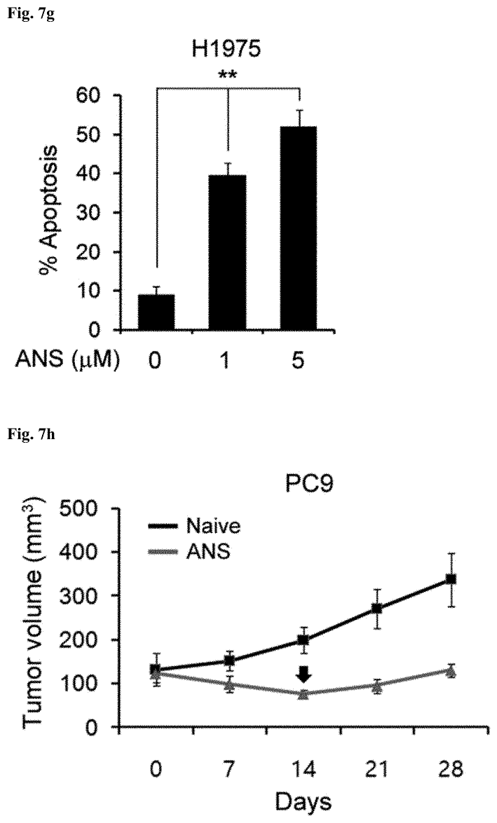

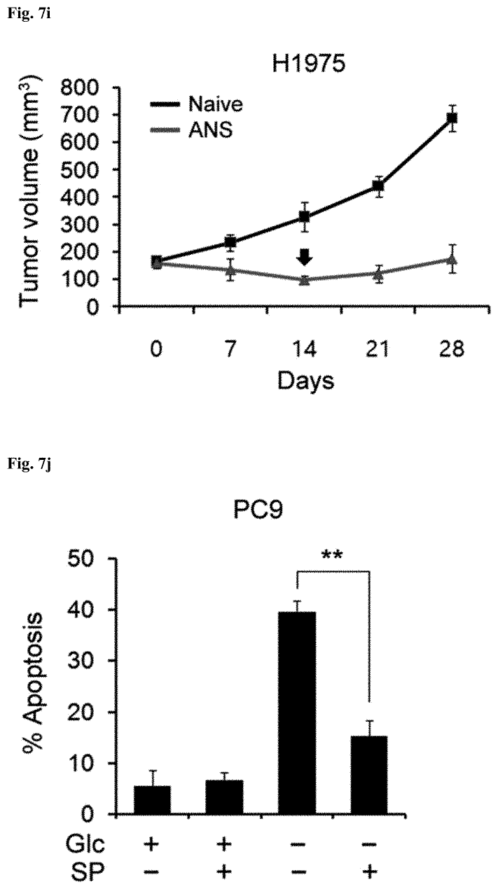

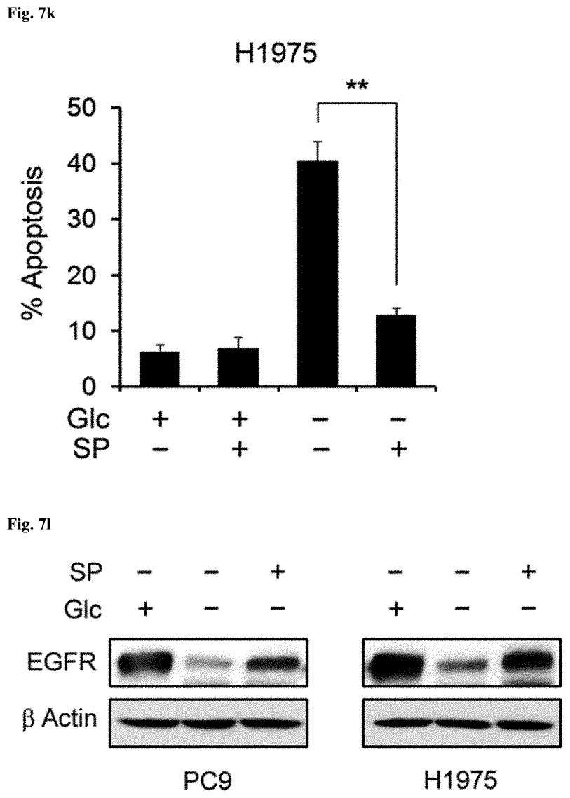

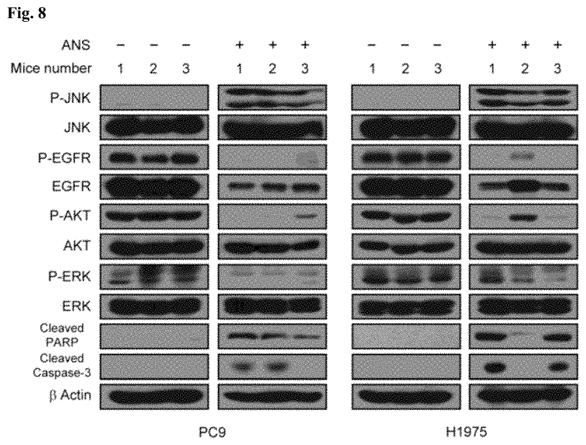

The downstream mechanism by which glucose-derived ATP production supports EGFR-mutant NSCLC survival was further investigated. JNK mediates apoptosis and cell death in response to environmental stress, but the mechanisms by which JNK activation induces tumor cell death remain unclear. Interestingly, JNK was strongly activated in EGFR-mutant NSCLCs in a time-dependent manner following glucose deprivation, but not glutamine deprivation (FIG. 7a). To examine whether glucose deprivation mediated JNK activation was due to inhibited mitochondrial ATP production, JNK activation upon rotenone treatment was assessed. As shown in FIG. 7b, rotenone treatment significantly induced JNK activation in both dose- and time-dependent manners. It was next examined whether ATP depletion-mediated JNK activation decreases EGFR levels. Consistent with the previous results, treatment with JNK-activator anisomycin significantly reduced EGFR levels, inhibited AKT and ERK phosphorylation, and led to PARP and caspase-3 cleavage in a dose-dependent manner (FIG. 7c). Anisomycin did not trigger apoptotic cell death in EGFR-WT NSCLCs (FIGS. 7d and 7e), whereas anisomycin-induced JNK activation led to significant induction of apoptosis in EGFR-mutant NSCLCs in a dose-dependent manner (FIGS. 7f and 7g) and robustly diminished growth in EGFR-mutant NSCLCs tumor (FIGS. 7h and 7i) via decreased EGFR levels (FIG. 8). These results indicate that glucose deprivation induced apoptotic cell death in EGFR-mutant NSCLCs only. The effects of JNK inhibition on glucose deprivation-induced apoptosis in EGFR-mutant NSCLCs were further examined to verify the requirement for JNK activation. Treatment with SP600125, a JNK inhibitor, significantly rescued cells from glucose deprivation-induced apoptosis (FIGS. 3j and 3k). Consistently, JNK inhibition significantly blocked the glucose deprivation-mediated reduction in EGFR levels (FIG. 7l). These results suggest that the ATP depletion-mediated JNK activation triggers apoptotic cell death in EGFR-mutant NSCLCs by reducing EGFR levels.

ROS Induces JNK-Mediated Reduction of EGFR Levels

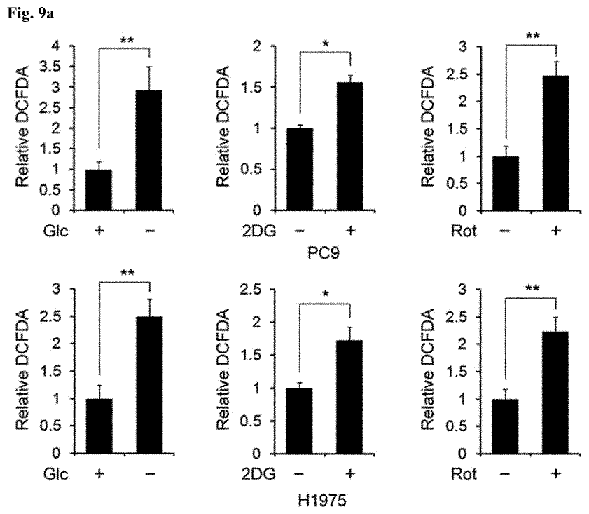

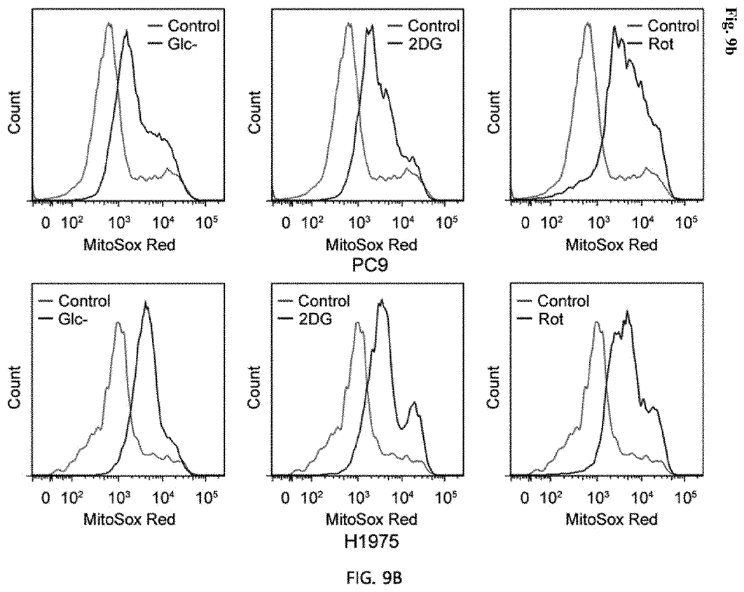

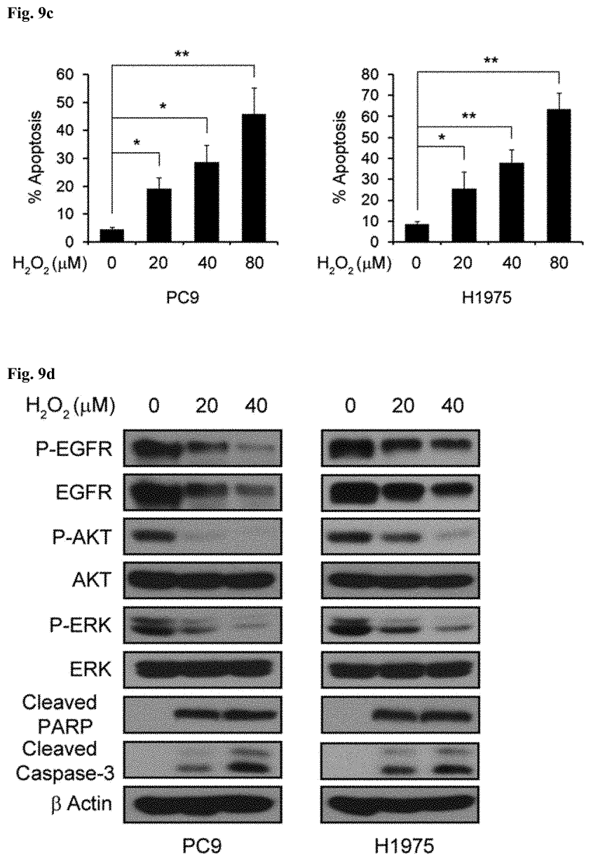

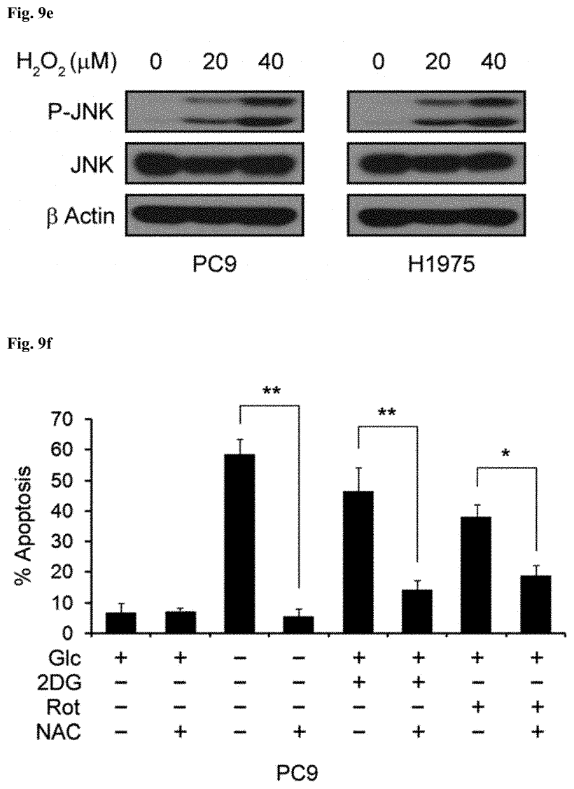

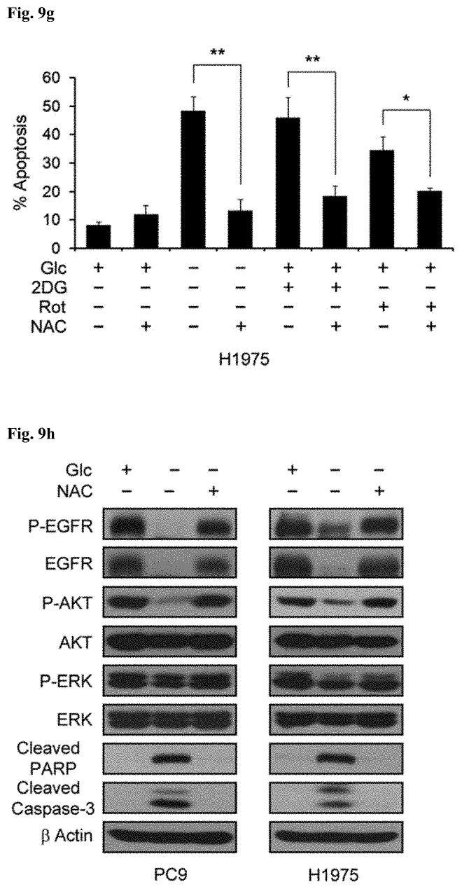

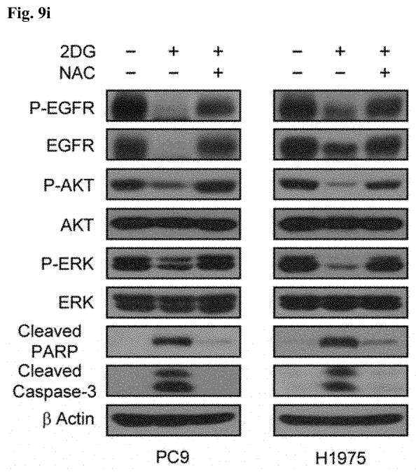

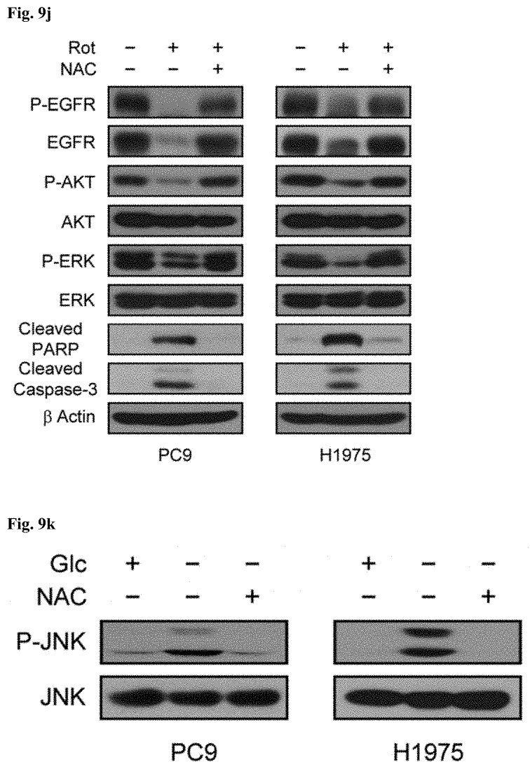

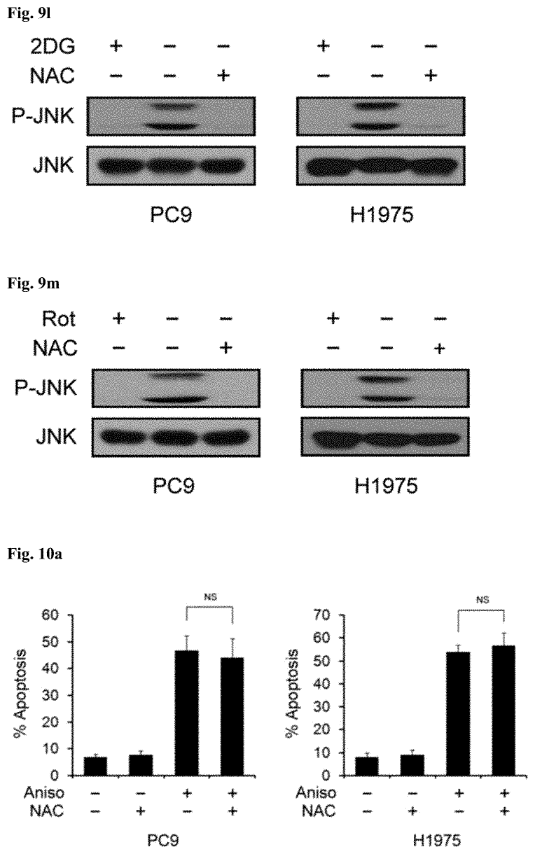

ATP production is a major function of the mitochondria, which provides a source of cellular ROS, and inhibition of mitochondrial respiration leads to excessive ROS. Considering the previous studies that showed that ROS activates mitogen-activated protein kinase (MAPK) and that ROS can induce JNK activation, it was speculated that the glucose deprivation-mediated mitochondrial ATP depletion might lead to a significant increase in ROS levels and JNK activation. Indeed, it was observed that glucose metabolism inhibition by glucose deprivation, 2DG treatment, or rotenone treatment led to significant increases in both cytoplasmic and mitochondrial ROS levels (FIGS. 9a and 9b). Also, hydrogen peroxide (H₂O₂) treatment activated apoptosis in EGFR-mutant NSCLCs in a dose-dependent manner (FIG. 9c).

To determine whether ROS accumulation would result in the JNK-mediated reduction of EGFR levels, the effects of ROS on EGFR signaling were tested. H₂O₂ treatment markedly reduced the EGFR levels, inhibited the AKT and ERK phosphorylation, and cleaved the PARP and caspase-3 in a dose-dependent manner (FIG. 9d). In addition, H₂O₂ activated JNK in a dose-dependent manner as well (FIG. 9e), indicating that ROS can activate apoptosis via JNK-mediated reduction of EGFR levels.

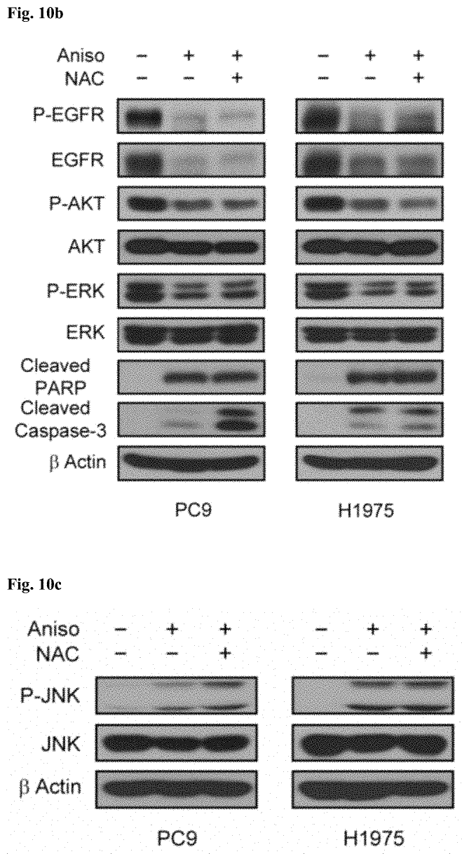

To confirm that the JNK-mediated reduction of EGFR levels was indeed due to ATP depletion-induced ROS production, apoptotic cell death upon glucose deprivation, 2DG, or rotenone treatment in the absence or presence of N-acetyl-L-cysteine (NAC) was assessed. NAC significantly rescued cell death caused by glucose deprivation, 2DG, or rotenone treatment (FIGS. 9f and 9g). Conversely, NAC did not rescue anisomycin-induced cell death (FIG. 10a), suggesting that JNK is a downstream target of ROS. NAC supplementation led to a significant recovery of EGFR levels and AKT and ERK phosphorylation following glucose deprivation, 2DG, or rotenone treatment; PARP and caspase-3 cleavage upon glucose deprivation, 2DG, or rotenone treatment was significantly inhibited by NAC (FIGS. 9h, 9i, and 9j). Importantly, JNK activation induced by glucose deprivation, 2DG, or rotenone treatment was completely inhibited by NAC (FIGS. 9k, 9l, and 9m). Conversely, NAC could not recover anisomycin-decreased EGFR signaling or EGFR levels (FIG. 10b) or inhibit anisomycin-induced JNK activation (FIG. 10c). Glucose metabolism may affect ROS levels through other mechanisms such as the imbalanced redox status. Verifying that inhibition of glucose-derived ATP generation in mitochondria is the major reason for ROS upregulation, it was tested whether glucose metabolism inhibition via glucose deprivation or 2DG treatment could affect NADP+/NADPH ratios through a compromised pentose phosphate pathway. Indeed, glucose metabolism inhibition had no significant effect on NADP+/NADPH ratios (data not shown), indicating that ATP depletion-mediated ROS generation induces JNK-mediated reduction of EGFR levels.

Autophagy is Required for JNK-Mediated Reduction of EGFR Levels

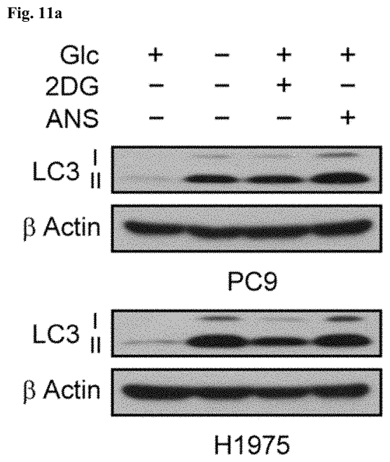

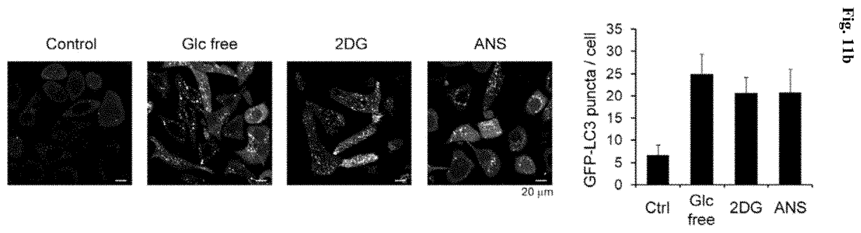

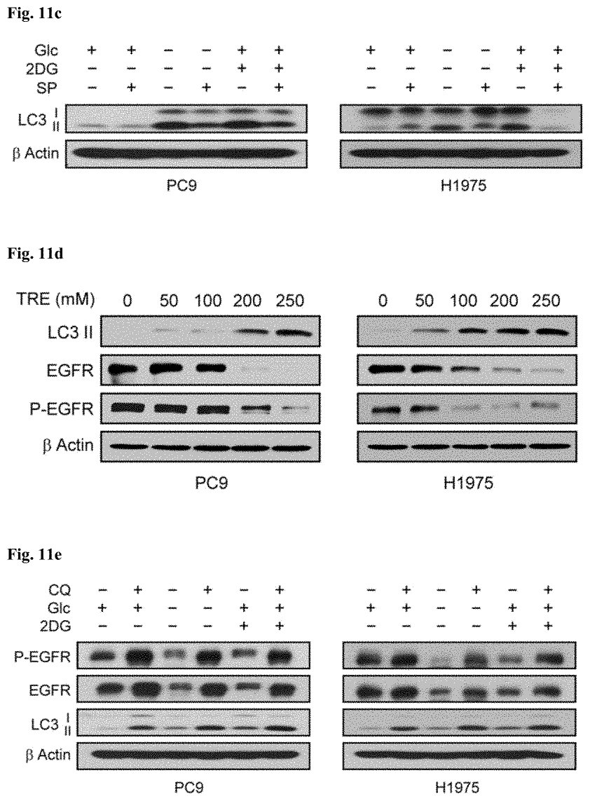

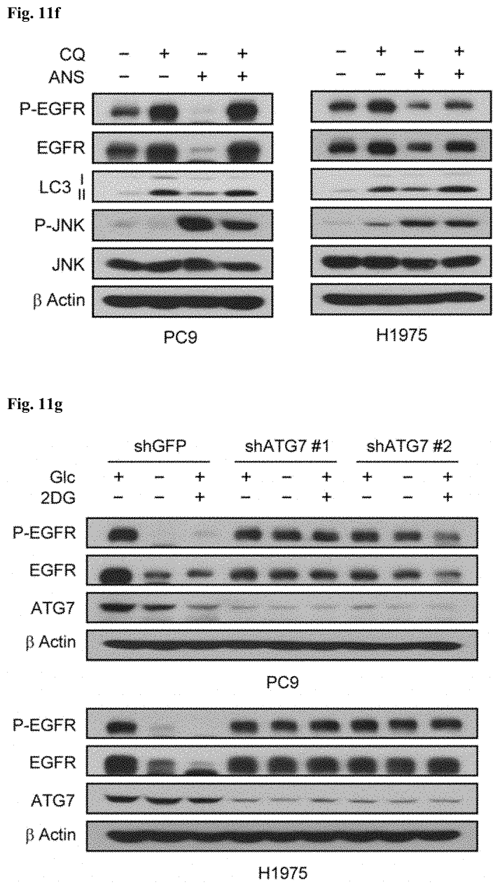

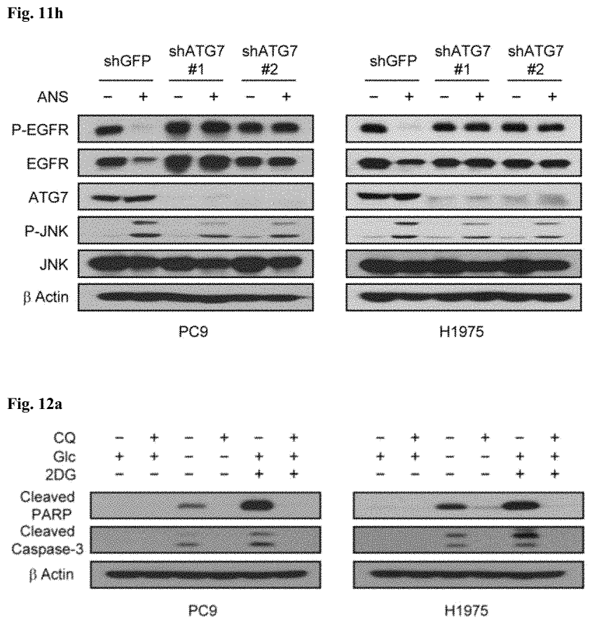

The mechanisms by which JNK regulates the EGFR expression were investigated. Either 2DG or anisomycin treatment had no significant effect on EGFR transcriptional level. The previous work of the present inventors demonstrated that activation of autophagy leads to EGFR degradation, which in turn induces apoptosis (So K S, Kim C H, Rho J K, Kim S Y, Choi Y J, Song J S, et al. Autophagosome-mediated EGFR down-regulation induced by the CK2 inhibitor enhances the efficacy of EGFR-TKI on EGFR-mutant lung cancer cells with resistance by T790 M. PloS one 2014; 9:e114000). Thus, it was tested whether JNK activates autophagy, which leads to EGFR degradation. Correspondingly, glucose deprivation, 2DG, or anisomycin treatment that significantly reduced EGFR levels resulted in a significant increase in LC3-II levels (FIG. 11a). As further confirmation of this increase in autophagic activity, GFP-LC3 reporter was used to examine the recruitment of LC3 into autophagosomes. As shown in FIG. 11b, the number of GFP-LC3 puncta profoundly increased upon glucose deprivation, 2DG, or anisomycin treatment compared with that in cells cultured in normal conditions.

To further confirm the role of JNK in glucose deprivation-mediated autophagy activation, LC3-II levels were assessed following glucose metabolism inhibition with or without SP600125. The increase in LC3-II levels induced by glucose deprivation or 2DG treatment was significantly inhibited by JNK inhibition (FIG. 11c), indicating that activated JNK induces autophagy activation.

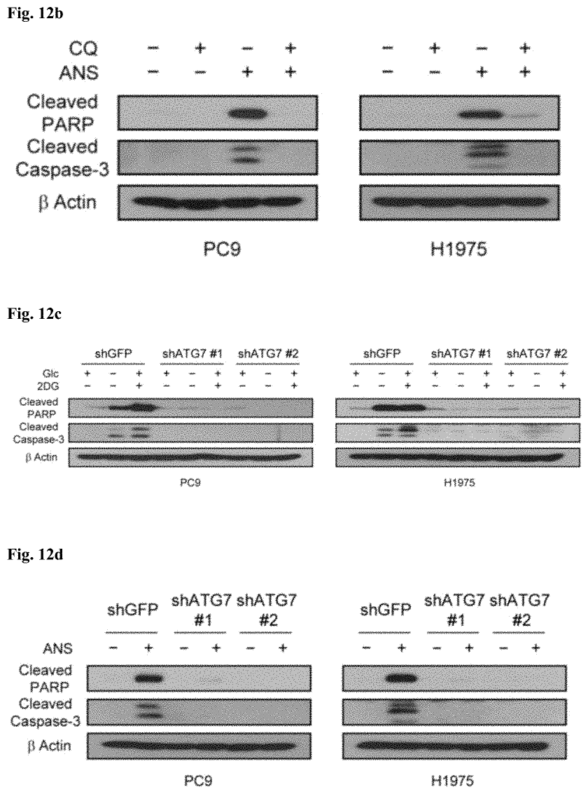

To test whether activated autophagy could induce EGFR degradation, the effect of autophagy activator on EGFR levels was examined. As shown in FIG. 11d, trehalose, which is known as an autophagy activator, significantly induced EGFR degradation in a dose-dependent manner. Moreover, the reduction in EGFR levels caused by glucose deprivation, 2DG, or anisomycin treatment was dramatically inhibited by chloroquine, a potent autophagy inhibitor (FIGS. 11e and 11f). In addition, glucose deprivation, 2DG, or anisomycin treatment did not reduce EGFR levels upon ATG7 knockdown (FIGS. 11g and 11h), suggesting that functional autophagy is required for JNK-mediated EGFR degradation. Given that autophagy is indispensable for glucose deprivation-mediated EGFR degradation, it was speculated that autophagy inhibition might suppress glucose deprivation-induced apoptotic cell death. Indeed, PARP and caspase-3 cleavage upon glucose deprivation, 2DG, or anisomycin treatment was robustly inhibited by chloroquine (FIGS. 12a and 12b). Consistently, PARP and caspase-3 cleavage was dramatically blocked upon ATG7 knockdown (FIGS. 12c and 12d). Thus, these results demonstrate that activated JNK mediates autophagy activation, which in turn induces EGFR degradation.

Targeting Glucose Metabolism Overcomes Acquired Resistance to EGFR-TKIs

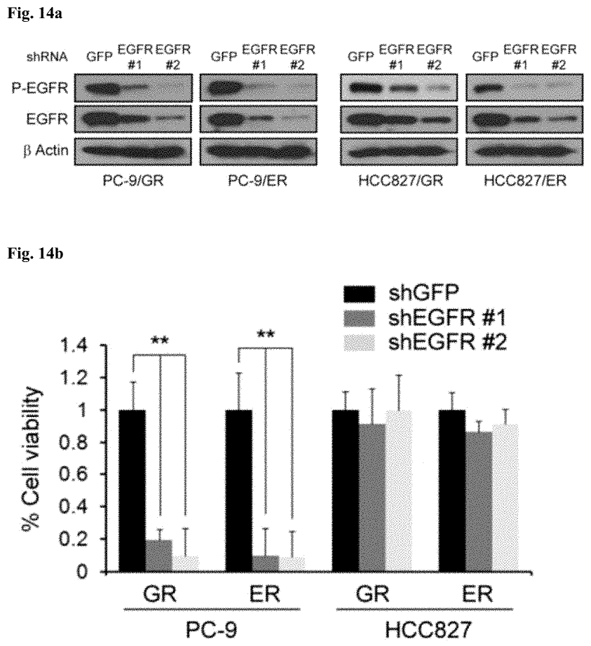

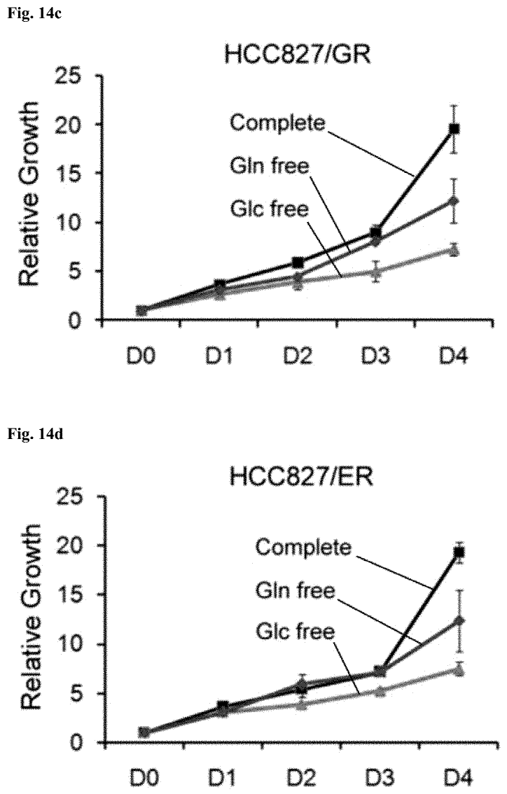

EGFR-TKI-resistant sublines were established in a previous study. The present inventors previously demonstrated that resistance in PC-9/GR and PC-9/ER cells is caused by a secondary T790 M mutation, whereas resistance in HCC827/GR and HCC827/ER cells is mediated by MET and AXL activation, respectively. The dependency of EGFR-TKIs-resistant cell lines on EGFR signaling varies depending on the mechanisms of acquired EGFR-TKI resistance. EGFR knockdown was used to evaluate the dependence of EGFR-TKI-resistant cell lines on EGFR signaling for proliferation. As shown in FIGS. 14a and 14b, no significant changes were observed in MET or AXL-mediated proliferation of resistant cells (CC827/GR and HCC827/ER), whereas EGFR knockdown markedly decreased T790 M-mediated proliferation of resistant cell (PC-9/GR and PC-9/ER).

Because glucose deprivation significantly decreased EGFR levels (FIGS. 1l and 1m), it was suspected this condition might affect PC-9/GR and PC-9/ER cell proliferation. The proliferation rates of both cell lines were significantly suppressed in response to glucose deprivation (FIGS. 13a and 13b), whereas glucose metabolism inhibition had minimal effect on the proliferation rates of HCC827/GR and HCC827/ER cells (FIGS. 14c and 14d). Glucose metabolism inhibition via glucose deprivation or 2DG treatment led to significant induction of apoptosis in cells with EGFR dependency such as PC-9/GR and PC-9/ER cells (FIGS. 13c to 13f).

Next, the involvement of JNK activation in glucose deprivation-mediated apoptotic death in PC-9/GR and PC-9/ER cells was investigated. As a result of this investigation, only glucose metabolism inhibition via glucose deprivation or 2DG treatment markedly activated JNK, resulting in EGFR level reduction, AKT and ERK phosphorylation inhibition, and PARP and caspase-3 cleavage in both PC-9/GR and PC-9/ER cells (FIG. 13g). The effects of glucose metabolism inhibition on EGFR signaling and apoptosis were also observed with anisomycin-mediated JNK activation (FIG. 13h). Anisomycin significantly induced apoptosis in a dose-dependent manner (FIGS. 13i and 13j).

Thus, these data demonstrate that EGFR signaling-dependent EGFR-TKI-resistant non-small cell lung cancer cells, that is, non-small cell lung cancer cells harboring T790 M mutation, can overcome acquired resistance to EGFR-TKIs via the inhibition of glucose metabolism or JNK activation.

Phosphorylated JNK is Reduced in NSCLC Tissues with EGFR Mutations

Based on preclinical data, the present inventors hypothesized that in patients with EGFR-mutant NSCLCs, the activity of JNK might be reduced to maintain EGFR-dependent tumor growth. To test this hypothesis and to evaluate our preclinical data, the phosphorylation of JNK and EGFR by IHC in 244 NSCLC tissues containing 126 EGFR-mutants and 118 WT EGFRs was examined.

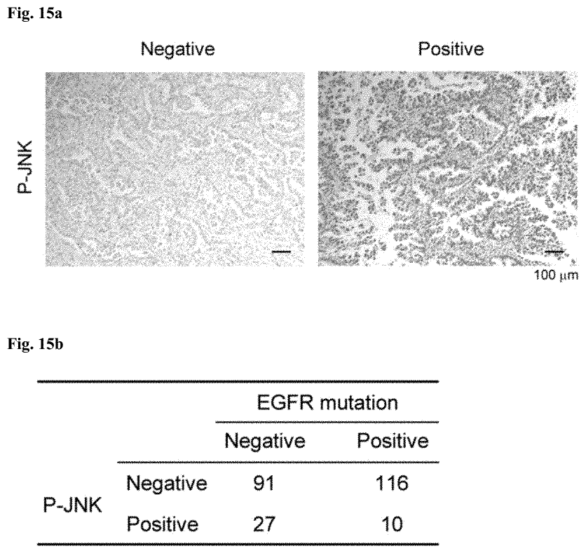

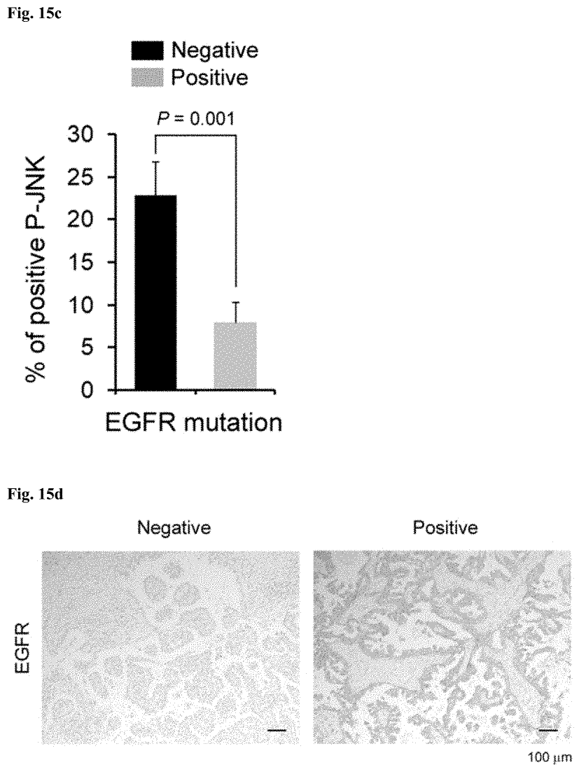

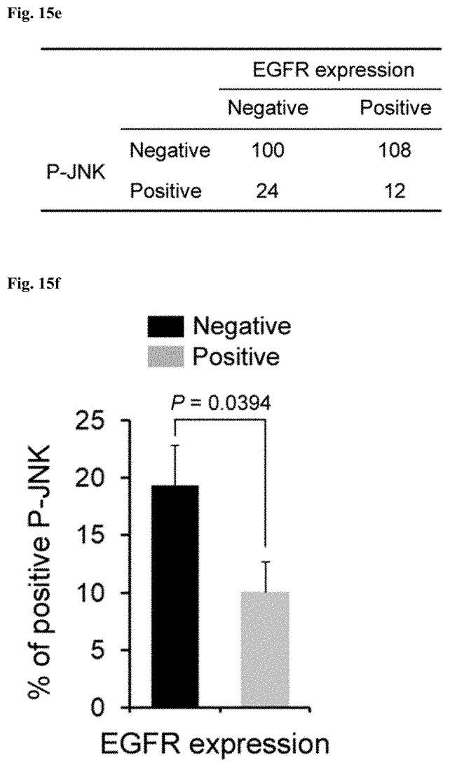

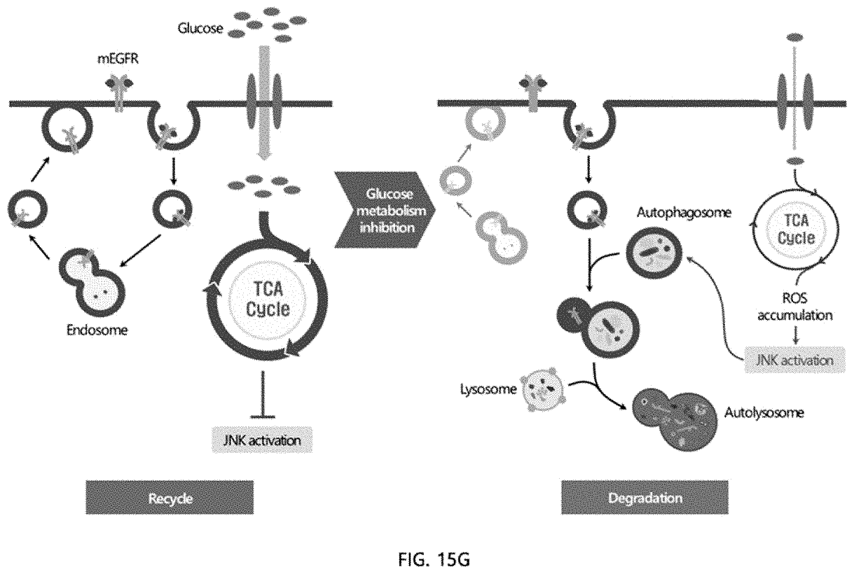

As shown in FIGS. 15a to 15c, the expression of phosphorylated JNK was found to be significantly decreased in EGFR-mutant NSCLCs (in 10 of 126, 7.94%) compared with WT EGFR (in 27 of 116, 23.28%).

In addition, a reverse correlation between phosphorylated JNK and EGFR expression in all tissues was observed regardless of EGFR mutation (FIGS. 15d to 15f). Thus, these data suggest that EGFR-mutant NSCLCs require reduced JNK activity to maintain EGFR-dependent tumor growth.

Collectively, these data reveal a novel dependence on EGFR-regulated enhanced glycolysis for maintaining EGFR levels in EGFR-mutant NSCLCs. Disruption of production of glucose-derived TCA cycle intermediates results in ROS-mediated JNK activation, leading to induction of autophagy-mediated EGFR degradation (FIG. 15g).

It will be understood by those skilled in the art that the invention can be implemented in other specific forms without changing the spirit or essential features of the invention. Therefore, it should be noted that the forgoing embodiments are merely illustrative in all aspects and are not to be construed as limiting the invention. The scope of the invention is defined by the appended claims rather than the detailed description of the invention. All changes or modifications or their equivalents made within the meanings and scope of the claims should be construed as falling within the scope of the invention.

What is claimed is:

1. A pharmaceutical composition for preventing or treating an EGFR-mutant non-small cell lung cancer comprising a c-Jun N-terminal kinase (JNK) activator as an active ingredient.

2. The pharmaceutical composition according to claim 1, wherein the JNK activator is anisomycin or a derivative thereof.

3. The pharmaceutical composition according to claim 1, wherein the non-small cell lung cancer harbors a deletion mutation in exon 19 of EGFR or a point mutation in exon 21 of EGFR.

4. The pharmaceutical composition according to claim 3, wherein the non-small cell lung cancer further harbors a T790 M mutation in exon 20 of EGFR.

5. The pharmaceutical composition according to claim 1, wherein the non-small cell lung cancer is resistant to gefitinib or erlotinib.

6. The pharmaceutical composition according to claim 1, wherein the non-small cell lung cancer is resistant to a reversible EGFR tyrosine kinase inhibitor due to a T790 M mutation in exon 20 of EGFR.

7. The pharmaceutical composition according to claim 1, wherein the JNK activator is present in an amount of 0.01 to 40% by weight, based on the total weight of the pharmaceutical composition.

8. The pharmaceutical composition according to claim 1, wherein the pharmaceutical composition is administered in an amount of 0.001 to 100 mg/kg/day.

9. The pharmaceutical composition according to claim 1, further comprising a pharmaceutically acceptable carrier, excipient or diluent.

10. The pharmaceutical composition according to claim 1, wherein the pharmaceutical composition is formulated into a liquid, powder, aerosol, injectable preparation, Ringer's solution, patch, capsule, pill, tablet, depot or suppository.

11. The pharmaceutical composition according to claim 1, wherein the non-small cell lung cancer is squamous cell carcinoma, adenocarcinoma, large cell carcinoma, adenosquamous carcinoma or sarcomatoid carcinoma.

12. A pharmaceutical composition for inhibiting the resistance of a non-small cell lung cancer to an EGFR tyrosine kinase inhibitor comprising a c-Jun N-terminal kinase (JNK) activator as an active ingredient.

13. The pharmaceutical composition according to claim 12, wherein the JNK activator is anisomycin.

14. The pharmaceutical composition according to claim 12, wherein the EGFR tyrosine kinase inhibitor is gefitinib or erlotinib.

15. A health functional food composition for preventing or ameliorating an EGFR-mutant non-small cell lung cancer comprising a c-Jun N-terminal kinase (JNK) activator as an active ingredient.

16. A method for treatment, amelioration, or prevention of an EGFR-mutant non-small cell lung cancer in a subject in need thereof, the method comprising administering to the subject a composition comprising a c-Jun N-terminal kinase (JNK) activator as an active ingredient.

17. The method of claim 16, wherein the composition is a pharmaceutical composition or health functional food composition.

18. The method of claim 17, wherein at least one of the following is true:
(i) the JNK activator is anisomycin or a derivative thereof;
(ii) the JNK activator is present in an amount of 0.01 to 40% by weight, based on the total weight of the pharmaceutical composition;
(iii) the pharmaceutical composition is administered in an amount of 0.001 to 100 mg/kg/day;
(iv) the pharmaceutical composition further comprises a pharmaceutically acceptable carrier, excipient or diluent; or
(v) the pharmaceutical composition is formulated into a liquid, powder, aerosol, injectable preparation, Ringer's solution, patch, capsule, pill, tablet, depot or suppository.

19. The method claim 16, wherein at least one of the following is true:
(i) the non-small cell lung cancer harbors a deletion mutation in exon 19 of EGFR or a point mutation in exon 21 of EGFR;
(ii) the non-small cell lung cancer harbors a T790 M mutation in exon 20 of EGFR;
(iii) the non-small cell lung cancer is resistant to gefitinib or erlotinib;
(iv) the non-small cell lung cancer is resistant to a reversible EGFR tyrosine kinase inhibitor due to a T790 M mutation in exon 20 of EGFR; or
(v) the non-small cell lung cancer is squamous cell carcinoma, adenocarcinoma, large cell carcinoma, adenosquamous carcinoma or sarcomatoid carcinoma.

20. A method for decreasing the resistance of a non-small cell lung cancer to an EGFR tyrosine kinase inhibitor in a subject in need thereof, the method comprising administering to the subject a composition comprising a c-Jun N-terminal kinase (JNK) activator as an active ingredient.
